(19) United States
(12) Patent Application Publication
(10) Pub. No.: US 2026/0234724 A1
(43) Pub. Date: Aug. 13, 2026

(54) COMPOSITIONS AND METHODS FOR IBD PATIENTS USING STOOL-DERIVED EUKARYOTIC NUCLEIC ACIDS

(71) Applicant: GENEOSCOPY, INC., St. Louis, MO (US)

(72) Inventors: Erica BARNELL, St. Louis, MO (US); Andrew BARNELL, St. Louis, MO (US); Elizabeth WURTZLER, St. Louis, MO (US); Ryan GHANNAM, St. Louis, MO (US); Richard ROBERTS, St. Louis, MO (US)

(21) Appl. No.: 19/150,769

(22) PCT Filed: Jan. 30, 2024
(86) PCT No.: PCT/US2024/013472

§ 371 Date: Jul. 24, 2025

Related U.S. Application Data
(60) Provisional application No. 63/441,973, filed on Jan. 30, 2023.
(60) Provisional application No. 63/544,876, filed on Oct. 19, 2023.

(51) Int. Cl. C12Q 1/6883

(52) U.S. Cl. CPC C12Q 1/6883; C12Q 2600/106; C12Q 2600/158

(57) ABSTRACT

The present disclosure provides compositions and methods for using stool-derived, eukaryotic, nucleic acid biomarkers to diagnose disease, assess disease activity, monitor mucosal healing, and predict therapeutic response. The described biomarkers can be used by practitioners to better diagnose, manage, and treat inflammatory bowel disease (IBD), including ulcerative colitis (UC) and Crohn's disease (CD).

### CROSS-REFERENCE TO RELATED APPLICATIONS

This application claims the benefit under 35 U.S.C. § 119 (e) of U.S. Provisional Application Ser. No. 63/441,973, filed on Jan. 30, 2023 and also of U.S. Provisional Application Ser. No. 63/544,876, filed on Oct. 19, 2023, the entire disclosures of both which are incorporated herein by reference.

### BACKGROUND AND SUMMARY

Inflammatory bowel disease (IBD) is a spectrum of difficult to manage disease states, including ulcerative colitis (UC) and Crohn's disease (CD). IBD afflicts over 3 million individuals in the United States and has an annual economic burden of over $6.3 billion. IBD typically requires a combination of multiple tests to make an initial diagnosis and can take many months to effectively treat. Generally, maintaining long-term remission is a goal of treatment in order to effectively avoid complications, surgery, malignancy, and iatrogenic side effects. Currently, methods for diagnosis, monitoring, and assessing therapeutic effectiveness in IBD patients primarily include invasive tests such as colonoscopy and sigmoidoscopy that require inconvenient and uncomfortable bowel preparation.

Existing noninvasive diagnostics within IBD fall into three categories: blood-based protein biomarkers, stool-based protein biomarkers, or stool-based microbiome biomarkers. Serology markers include Saccharomyces cerevisiae mannan antibodies, perinuclear anti-neutrophil cytoplasmic antibody, and IgA/IgG antibodies. Fecal markers include calprotectin, lactoferrin, and lymphocyte markers. Stool-based microbiome biomarkers have been investigated but no definitive panels are currently available. However, the current intended use, sensitivity, and specificity of these noninvasive diagnostic tests are insufficient to assist physicians with accurately diagnosing patients in a timely manner, monitoring inflammation and mucosal healing during treatment, and predicting response to therapeutics. Thus, there exists a need for new compositions and methods for providing clinicians with predictive and clinical biomarkers for IBD.

Accordingly, the present disclosure provides compositions and methods for using stool-derived, eukaryotic, nucleic acid biomarkers to diagnose disease, assess disease activity, monitor mucosal healing, and predict therapeutic response. Ultimately, the described biomarkers can be used by practitioners to better diagnose, manage, and treat IBD.

The compositions and methods of the present disclosure provide several benefits compared to the current state of the art. For example, the present disclosure utilizes extraction methods that permits the isolation of high-quality eukaryotic nucleic acids (e.g., DNA and/or RNA) and proteins (e.g., calprotectin) from a stool sample. Methods that can be utilized according to the present disclosure are described in PCT International Application Publication WO 2018/081580 and also in PCT International Application Publication WO 2019/232483, both of which are herein incorporated by reference in their entirety. For instance, stool-derived eukaryotic RNA (seRNA) is provided herein to specify the eukaryotic RNA preserved during the process of fecal matter generation, and which is subsequently extracted from stool samples by the described methods.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTIONS OF THE DRAWINGS

The detailed description particularly refers to the accompanying figures in which:

FIGS. 1A-1B show seRNA classifiers to approximate disease activity as it relates to remission versus active disease (FIG. 1A) and disease severity as it relates to differentiating mild versus moderate disease (FIG. 1B).

FIGS. 2A-2C show seRNA biomarkers can be used to predict response to targeted therapeutics, as demonstrated by the longitudinal seRNA assessment of 16 subjects being treated with targeted therapies. FIG. 2A shows subjects treated with vedolizumab. FIG. 2B shows subjects treated with ustekinumab. FIG. 2C shows subjects treated with infliximab.

FIG. 3 shows protein-based calprotectin measurements (ELISA assay) compared to RNA-based calprotectin measurements (whole transcriptome sequencing).

FIG. 4 shows the receiver operator characteristic (ROC) comparing the protein-based method and the RNA-based method for calprotectin.

### DETAILED DESCRIPTION

Various embodiments of the invention are described herein as follows. In an illustrative aspect, a method of assessing disease in a subject is provided. The method comprises the steps of measuring the level of expression of one or more stool-derived eukaryotic nucleic acid biomarkers selected from the biomarkers described herein in eukaryotic nucleic acid extracted from a stool sample from the subject, wherein the measured expression level of the one or more stool-derived eukaryotic nucleic acid biomarkers in the stool sample indicates the assessment of disease in the subject.

In an embodiment, the method comprises comparing the measured expression level of the one or more stool-derived eukaryotic nucleic acid biomarkers in the stool sample with the measured expression level of the one or more stool-derived eukaryotic nucleic acid biomarkers in a control, wherein a difference in the measured expression level of the one or more stool-derived eukaryotic nucleic acid biomarkers in the stool sample relative to the measured expression level of the one or more stool-derived eukaryotic nucleic acid biomarkers in the control indicates the assessment of disease in the subject.

In an embodiment, the nucleic acid is DNA. In an embodiment, the nucleic acid is RNA. In an embodiment, the nucleic acid is a combination of DNA and RNA.

In an embodiment, the disease is inflammatory bowel disease (IBD). In an embodiment, the disease is ulcerative colitis (UC). In an embodiment, the disease is Crohn's disease (CD).

In an embodiment, the assessment of disease comprises an assessment of disease activity in the subject. In an embodiment, the assessment is a determination of active IBD symptoms in the subject. In an embodiment, the determination of active IBD symptoms indicates mild IBD symptoms. In an embodiment, the determination of active IBD symptoms indicates moderate IBD symptoms. In an embodiment, the determination of active IBD symptoms indicates severe IBD symptoms. In an embodiment, the assessment is a determination of IBD remission in the subject.

The present disclosure includes various stool-derived eukaryotic nucleic acid biomarkers. Certain stool-derived eukaryotic nucleic acid biomarkers are provided in Tables 1-5 of the present disclosure. As described herein, stool-derived eukaryotic nucleic acid biomarkers can be selected from one of the Tables, for example a grouping of 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers. Nucleic acids of the invention can include nucleic acids having a nucleotide sequence of any one of the stool-derived eukaryotic nucleic acid biomarkers listed in Table 1 or Table 2 or Table 3 or Table 4, or a combination of Table 1 and Table 2 or in Table 3 or Table 4, or a combination provided in Table 5, or a nucleic acid sequence that is at least about 70%, at least about 75%, at least about 80%, at least about 85%, at least about 90%, at least about 95%, at least about 99% identical to a nucleic acid sequence of any one of the stool-derived eukaryotic nucleic acid biomarkers listed in Table 1 or Table 2 or Table 3 or Table 4, or a combination of Table 1 and Table 2 or in Table 3 or Table 4, or a combination provided in Table 5.

The grouping of biomarkers from one of the Tables can be derived based on one or more parameters such as IBD disease severity (e.g., mild, moderate, active, remission) and patient response. Moreover, the grouping of biomarkers can be derived based on frequency, error rate, and/or combination potential.

In an embodiment, the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 1. In an embodiment, the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 2. In an embodiment, the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 3. In an embodiment, the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 4. In an embodiment, the stool-derived eukaryotic nucleic acid biomarkers comprise a combination of biomarkers listed in Table 5.

TABLE 1
Various Biomarkers

| | | | |
|---|---|---|---|
| APOA4 | ENSG00000290010.1 | RPS26P31 | TRAF3IP2-AS1 |
| EZR | ENSG00000290038.1 | H3P6 | TRAF3IP1 |
| LITAF | SOD2 | H3P36 | LILRB4 |
| ABCG2 | SPATA31A6 | FTH1P4 | TGFB1 |
| NXPE4 | USP17L15 | NEAT1 | TGFB2 |
| TIMP1 | USP17L20 | ENSG00000248223.1 | TGFB3 |
| MAPK3 | USP17L18 | ENSG00000280136.2 | IL21 |
| RNASET2 | ELOA3DP | ENSG00000289136.1 | IL16 |
| TMED9 | FABP6 | LRRC70 | IL21R |
| SYNGR2 | PHLDA2 | MT.RNR2 | FAT4 |
| CEACAM5 | IGLC3 | MT.RNR1 | HRH4 |
| GCNT3 | APOA1.AS | ENSG00000281383.1 | TMEM71 |
| CD74 | ENSG00000249406.3 | SI | ENSG00000268509.2 |
| GSTA1 | ENSG00000285513.1 | UACA | CD28 |
| ANXA2 | ZFHX4 | ZMAT1 | CD3D |
| FOXO3 | PRB2 | FILIP1L | CD3G |
| IL2RG | MAP1B | KRT13 | CD5 |
| FOSL2 | ZAN | ZNG1DP | CD6 |
| CA1 | ABI3BP | SLC25A51 | CHRM3-AS2 |
| PLAUR | IGFN1 | DONSON | CTLA4 |
| SKP1 | MUC7 | ALPI | FLT3LG |
| UPP1 | OPRPN | ENSG00000276490.1 | MAL |
| SAT1 | KRT9 | CHMP4BP1 | MGC40069 |
| NFKBIA | ANKRD20A4P | RPS26 | PBX4 |
| ITM2C | SSC5D | H2BP2 | SIRPG |
| PARM1 | ANKRD20A2P | ENSG00000243403.1 | THEMIS |
| ZFP36L1 | RIMBP3C | H2AC19 | TRAT1 |
| B2M | CACNA1H | PSMB8.AS1 | BANK1 |
| ITLN1 | ENSG00000240240.10 | GABBR1 | CD19 |
| NUPR1 | PRB1 | HLA.A | CD22 |
| SERPINA1 | ANKRD20A1 | UBD | CD79A |
| LCP2 | RIMBP3B | ENSG00000227766.1 | CR2 |
| PRDX5 | RIMBP3 | OR2I1P | FCRL2 |
| PIGR | SPATA31A7 | HBB | IGKC |
| RPL19 | ANKRD20A3P | MTTP | PAX5 |
| PI3 | SPATA31A5 | KRT1 | CD160 |
| HCAR3 | ANKRD20A5P.1 | EFCAB5 | KIR2DL1 |
| FP36L1 | ENSG00000291287.1 | ALAS2 | KIR2DL3 |
| PDLIM7 | SRGN | HLA.DQA1 | KIR2DLA |
| HCAR2 | S100A8 | UTY | KIR3DL1 |
| GLRX | TMSB4XP8 | TRIM31 | KIR3DS1 |
| MUC12 | GPSM3 | SPRR3 | NCR1 |
| IL1B | EEF1A1P6 | SLC6A14 | PTGDR |
| COL1A1 | FTH1P11 | SLC5A3 | SH2D1B |
| TRAF3IP2 | FTH1P23 | RPS4Y1 | ADAP2 |
| HLA.E | CCL4L2 | ENSG00000280800.1 | CSF1R |
| TSPO | CCL3L1 | DEFA1 | FPR3 |
| AREG | ENSG00000279198.2 | DDX3Y | KYNU |
| G0S2 | ENSG00000288681.1 | DDAH2 | PLA2G7 |
| PARK7 | ENSG00000290018.1 | CEACAM8 | RASSF4 |
| IP6K2 | TRIP11 | AHNAK | TFEC |
| FOS | DGCR8 | VEPH1 | CD1A |
| APOA1 | BLNK | USP17L26 | CD1B |
| PILRA | ERMN | SLC26A2 | CD1E |
| LST1 | MS4A1 | SETD3 | CLEC10A |
| APOC3 | CXCL5 | RESF1 | CLIC2 |
| CREB3L3 | TAF9B | PTX3 | WFDC21P |
| TRIM65 | HLA.DRB5 | PTMAP2 | CA4 |
| BCL6 | USP17L6P | PTMA | CEACAM3 |
| CCDC144A | USP17L5 | PRR20C | CXCR1 |
| TRAF3IP3 | USP17L12 | PRR20B | CXCR2 |
| MUCL3 | USP17L30 | PRR20A | CYP4F3 |
| CFD | USP17L25 | PHLDA1.AS1 | FCGR3B |
| USP17L9P | USP17L10 | NUFIP2 | HAL |
| FABP2 | USP17L29 | MUC20P1 | KCNJ15 |
| DEFA6 | USP17L24 | MUC20 | MEGF9 |
| CALD1 | USP17L13 | MORF4L1P1 | SLC25A37 |
| HSPA1B | USP17L11 | MANSC1 | STEAP4 |
| RPL41P5 | USP17L27 | EPPK1 | TECPR2 |
| ENSG00000290971.1 | CT47A3 | ENSG00000281181.1 | TLE3 |
| NIBAN2 | USP17L19 | ENSG00000280614.1 | TNFRSF10C |
| ENSG00000271581.1 | USP17L22 | ENSG00000255224.2 | VNN3 |
| ALKBH7 | USP17L17 | ELOA3P | ACVRL1 |
| MUC3A | USP17L14P | CD68 | APLN |
| HLA.C | USP17L21 | AQP8 | BCL6B |
| SLC6A4 | USP17L16P | TMTC1 | BMP6 |
| NXPE1 | USP17L23 | SRSF4 | BMX |
| MAF | GFY | PNN | CDH5 |
| BCL2A1 | PLAC4 | MUC17 | CLEC14A |
| GPX2 | ENSG00000287037.1 | GADD45A | DIPK2B |
| HSPA1A | APOB | ADGRB2 | ADGRL4 |
| PROK2 | PHLDA1 | ENSG00000289901.1 | EMCN |
| SH3RF3 | COL6A2 | NOS2 | ESAM |
| ACY1 | CYP4F2 | SPINK5 | ESM1 |
| NAMPT | MMP1 | VCX3B | FAM124B |
| ENPEP | IGLC1 | POLR2M | HECW2 |
| HLA | IGLC2 | TRAPPC5 | HHIP |
| CST7 | MUC5AC | RXFP4 | KDR |
| CARD16 | IGLL5 | TRIM72 | MMRN1 |
| CXCL8 | ATP5ME | TDRD6 | MMRN2 |
| TM4SF1 | RPL21P16 | AFAP1L1 | MYCT1 |
| HLA.DRB1 | RGS2 | LAMTOR5 | PALMD |
| ACTB | H3.3B | KIF17 | PEAR1 |
| FTL | H3.3A | GAGE12J | PGF |
| ZFP36 | H3P16 | TMEM70 | PLXNA2 |
| IER3 | H3P47 | DBNDD1 | PTPRB |
| UBC | POTEE | SOD3 | ROBO4 |
| S100A9 | H3.5 | RHOD | SHANK3 |
| BEST1 | RPS26P8 | KCNC4 | SHE |
| FTH1 | RPS26P6 | SPRR2E | TEK |
| RPS9 | FTH1P12 | BMP8A | TIE1 |
| ACTG1 | FTH1P3 | TTLL8 | COL3A1 |
| HBA2 | ACTBP2 | IFNA7 | COL6A1 |
| MUC2 | FTH1P8 | FYB2 | DCN |
| HBA1 | FTH1P10 | GRIK3 | GREM1 |
| ENSG00000225864.1 | RPS26P15 | LGALS4 | PAMR1 |
| RPL41P1 | RPS26P58 | NR1D1 | TAGLN |
| RPL41 | FTH1P20 | STXBP3 | PDX1 |
| FTH1P2 | FTLP3 | HLA-DRA | GAST |
| ENSG00000256249.1 | NAMPTP1 | KLF4 | TFF2 |
| ENSG00000264281.3 | TMSB4XP6 | CD177 | TFF1 |
| TPM3P5 | FTH1P5 | NOD2 | PGC |
| ENSG00000279483.2 | RPS27AP5 | TREX1 | MUC6 |
| ENSG00000285565.1 | FTH1P7 | FCER1A | KRT18 |
| ENSG00000288706.1 | RPS26P47 | STARD7 | KRT8 |

TABLE 2
Biomarkers Possibly Associated with Disease Severity

| | | |
|---|---|---|
| APOA4 | LCP2 | CFD |
| EZR | PRDX5 | USP17L9P |
| LITAF | PIGR | FABP2 |
| ABCG2 | RPL19 | DEFA6 |
| NXPE4 | PI3 | CALD1 |
| TIMP1 | HCAR3 | HSPA1B |
| MAPK3 | FP36L1 | RPL41P5 |
| RNASET2 | PDLIM7 | ENSG00000290971.1 |
| TMED9 | HCAR2 | NIBAN2 |
| SYNGR2 | GLRX | ENSG00000271581.1 |
| CEACAM5 | MUC12 | ALKBH7 |
| GCNT3 | IL1B | MUC3A |
| CD74 | COL1A1 | HLA.C |
| GSTA1 | TRAF3IP2 | SLC6A4 |
| ANXA2 | HLA.E | NXPE1 |
| FOXO3 | TSPO | MAF |
| IL2RG | AREG | BCL2A1 |
| FOSL2 | G0S2 | GPX2 |
| CA1 | PARK7 | HSPA1A |
| PLAUR | IP6K2 | PROK2 |
| SKP1 | FOS | SH3RF3 |
| UPP1 | APOA1 | ACY1 |
| SAT1 | PILRA | NAMPT |
| NFKBIA | LST1 | ENPEP |
| ITM2C | APOC3 | HLA |
| PARM1 | CREB3L3 | CST7 |
| ZFP36L1 | TRIM65 | CARD16 |
| B2M | BCL6 | CXCL8 |
| ITLN1 | CCDC144A | TM4SF1 |
| NUPR1 | TRAF3IP3 | HLA.DRB1 |
| SERPINA1 | MUCL3 | |

TABLE 3
Biomarkers Possibly Associated with Prediction of Response

| | | |
|---|---|---|
| MUC12 | USP17L21 | PTMA |
| ACTB | USP17L16P | PRR20C |
| FTL | USP17L23 | PRR20B |
| NFKBIA | USP17L9P | PRR20A |
| G0S2 | GFY | PHLDA1.AS1 |
| ZFP36 | PLAC4 | PARK7 |
| IER3 | ENSG00000287037.1 | NUFIP2 |
| UBC | SH3RF3 | MUC20P1 |
| S100A9 | APOB | MUC20 |
| B2M | COL1A1 | MORF4L1P1 |
| BEST1 | APOC3 | MANSC1 |
| FTH1 | CALD1 | IP6K2 |
| CXCL8 | PHLDA1 | EPPK1 |
| FOS | COL6A2 | ENSG00000281181.1 |
| RPS9 | CYP4F2 | ENSG00000280614.1 |
| ACTG1 | MMP1 | ENSG00000271581.1 |
| HBA2 | IGLC1 | ENSG00000255224.2 |
| MUC2 | IGLC2 | ELOA3P |
| HSPA1B | MUC5AC | CST7 |
| HSPA1A | IGLL5 | CD68 |
| HLA.E | ATP5ME | AQP8 |
| HBA1 | RPL21P16 | TMTC1 |
| ENSG00000225864.1 | PLAUR | SRSF4 |
| RPL41P1 | NAMPT | PNN |
| RPL41 | RGS2 | MUC17 |
| FTH1P2 | SAT1 | GADD45A |
| ENSG00000256249.1 | H3.3B | ADGRB2 |
| RPL41P5 | BCL2A1 | ENSG00000289901.1 |
| ENSG00000264281.3 | H3.3A | NOS2 |
| TPM3P5 | H3P16 | PILRA |
| ENSG00000279483.2 | H3P47 | SLC6A4 |
| ENSG00000285565.1 | HCAR2 | SPINK5 |
| ENSG00000288706.1 | POTEE | VCX3B |
| ENSG00000290010.1 | H3.5 | POLR2M |
| ENSG00000290038.1 | RPS26P8 | PDLIM7 |
| SOD2 | RPS26P6 | TRAPPC5 |
| SPATA31A6 | FTH1P12 | RXFP4 |
| USP17L15 | FTH1P3 | TRIM72 |
| USP17L20 | ACTBP2 | TDRD6 |
| USP17L18 | FTH1P8 | AFAP1L1 |
| ELOA3DP | FTH1P10 | TRIM65 |
| APOA4 | RPS26P15 | LAMTOR5 |
| APOA1 | RPS26P58 | KIF17 |
| DEFA6 | FTH1P20 | GAGE12J |
| FABP6 | FTLP3 | TMEM70 |
| MAF | NAMPTP1 | DBNDD1 |
| PHLDA2 | TMSB4XP6 | SOD3 |
| IGLC3 | FTH1P5 | RHOD |
| APOA1.AS | RPS27AP5 | ALKBH7 |
| ENSG00000249406.3 | FTH1P7 | KCNC4 |
| ENSG00000285513.1 | RPS26P47 | SPRR2E |
| ZFHX4 | RPS26P31 | BMP8A |
| PRB2 | H3P6 | TTLL8 |
| MAP1B | H3P36 | IFNA7 |
| ZAN | FTH1P4 | FYB2 |
| ABI3BP | NEAT1 | GRIK3 |
| IGFN1 | ENSG00000248223.1 | LGALS4 |
| CCDC144A | HCAR3 | NR1D1 |
| MUC7 | ENSG00000280136.2 | STXBP3 |
| OPRPN | ENSG00000289136.1 | ABCG2 |
| KRT9 | LRRC70 | NXPE1 |
| ANKRD20A4P | MT.RNR2 | NXPE4 |
| SSC5D | MT.RNR1 | HLA-DRA |
| ANKRD20A2P | ENSG00000281383.1 | TIMP1 |
| RIMBP3C | CREB3L3 | FOXO3 |
| CACNA1H | SI | CA1 |
| ENSG00000240240.10 | UACA | CD74 |
| PRB1 | FABP2 | IL2RG |
| ANKRD20A1 | ZMAT1 | RNASET2 |
| RIMBP3B | FILIP1L | GSTA1 |
| RIMBP3 | KRT13 | ANXA2 |
| SPATA31A7 | ZNG1DP | UPP1 |
| ANKRD20A3P | SLC25A51 | SKP1 |
| SPATA31A5 | DONSON | GCNT3 |
| ENSG00000290971.1 | ALPI | GLRX |
| ANKRD20A5P.1 | ENSG00000276490.1 | SYNGR2 |
| ENSG00000291287.1 | CHMP4BP1 | LCP2 |
| MUCL3 | PROK2 | FOSL2 |
| SRGN | RPS26 | KLF4 |
| IL1B | LST1 | ZFP36L1 |
| S100A8 | H2BP2 | ITM2C |
| TMSB4XP8 | ENSG00000243403.1 | PARM1 |
| CFD | H2AC19 | TMED9 |
| GPSM3 | PSMB8.AS1 | ACY1 |
| EEF1A1P6 | HLA.C | GPX2 |
| FTH1P11 | GABBR1 | PRDX5 |
| FTH1P23 | HLA.A | CARD16 |
| CCL4L2 | UBD | MAPK3 |
| CCL3L1 | ENSG00000227766.1 | EZR |
| ENSG00000279198.2 | OR2I1P | NUPR1 |
| ENSG00000288681.1 | HBB | CD177 |
| ENSG00000290018.1 | ENPEP | LITAF |
| TSPO | MTTP | SERPINA1 |
| TRIP11 | KRT1 | AREG |
| DGCR8 | EFCAB5 | TM4SF1 |
| BLNK | ALAS2 | ITLN1 |
| ERMN | HLA.DQA1 | NOD2 |
| MS4A1 | UTY | TREX1 |
| CXCL5 | TRIM31 | BCL6 |
| TAF9B | SPRR3 | FCER1A |
| HLA.DRB1 | SLC6A14 | STARD7 |
| HLA.DRB5 | SLC5A3 | TRAF3IP2-AS1 |
| USP17L6P | RPS4Y1 | TRAF3IP1 |
| USP17L5 | NIBAN2 | TRAF3IP2 |
| USP17L12 | MUC3A | TRAF3IP3 |
| USP17L30 | ENSG00000280800.1 | LILRB4 |
| USP17L25 | DEFA1 | TGFB1 |
| USP17L10 | DDX3Y | TGFB2 |
| USP17L29 | DDAH2 | TGFB3 |
| USP17L24 | CEACAM8 | IL21 |
| USP17L13 | AHNAK | IL16 |
| USP17L11 | VEPH1 | IL21R |
| USP17L27 | USP17L26 | FAT4 |
| CT47A3 | SLC26A2 | HRH4 |
| USP17L19 | SETD3 | TMEM71 |
| USP17L22 | RESF1 | ENSG00000268509.2 |
| USP17L17 | PTX3 | |
| USP17L14P | PTMAP2 | |

TABLE 4
Biomarkers Possibly Associated with Cell Deconvolution

| | | |
|---|---|---|
| CD28 | PLA2G7 | FAM124B |
| CD3D | RASSF4 | HECW2 |
| CD3G | TFEC | HHIP |
| CD5 | CD1A | KDR |
| CD6 | CD1B | MMRN1 |
| CHRM3-AS2 | CD1E | MMRN2 |
| CTLA4 | CLEC10A | MYCT1 |
| FLT3LG | CLIC2 | PALMD |
| MAL | WFDC21P | PEAR1 |
| MGC40069 | CA4 | PGF |
| PBX4 | CEACAM3 | PLXNA2 |
| SIRPG | CXCR1 | PTPRB |
| THEMIS | CXCR2 | ROBO4 |
| TRAT1 | CYP4F3 | SHANK3 |
| BANK1 | FCGR3B | SHE |
| CD19 | HAL | TEK |
| CD22 | KCNJ15 | TIE1 |
| CD79A | MEGF9 | VEPH1 |
| CR2 | SLC25A37 | COL1A1 |
| FCRL2 | STEAP4 | COL3A1 |
| IGKC | TECPR2 | COL6A1 |
| MS4A1 | TLE3 | COL6A2 |
| PAX5 | TNFRSF10C | DCN |
| CD160 | VNN3 | GREM1 |
| KIR2DL1 | ACVRL1 | PAMR1 |
| KIR2DL3 | APLN | TAGLN |
| KIR2DLA | BCL6B | MUC2 |
| KIR3DL1 | BMP6 | PDX1 |
| KIR3DS1 | BMX | GAST |
| NCR1 | CDH5 | MUC5AC |
| PTGDR | CLEC14A | TFF2 |
| SH2D1B | DIPK2B | TFF1 |
| ADAP2 | ADGRL4 | PGC |
| CSF1R | EMCN | MUC6 |
| FPR3 | ESAM | KRT18 |
| KYNU | ESM1 | KRT8 |

TABLE 5
Combination of Biomarkers

| Combination | Biomarkers |
|---|---|
| A | APOA4 and/or EZR and/or LITAF and/or ABCG2 and/or NXPE4 and/or TMP1 and/or |
| | FOXO3 and/or CD74 and/or ANXA2 and/or FOSL2 and/or MAPK3 and/or RNASET2 and/or |
| | CEACAM5 and/or TMED9 |
| B | SKP1 and/or UPP1 and/or SAT1 and/or NFKBIA and/or ITM2C and/or ZFP36L1 and/or |
| | PLAUR and/or ANXA2 and/or B2M and/or APOA4 |
| C | ITGA4 and/or ITGB7 |
| D | IL12A and/or IL12B and/or IL23A |
| E | TNFRSF1A and/or TNFRSF1B |

In an embodiment, the one or more stool-derived eukaryotic nucleic acid biomarkers comprise stool-derived eukaryotic RNA biomarkers. In an embodiment, the stool-derived eukaryotic RNA biomarkers comprise enterocyte-specific biomarkers. In an embodiment, the stool-derived eukaryotic RNA biomarkers consist essentially of enterocyte-specific biomarkers. In an embodiment, the stool-derived eukaryotic RNA biomarkers consist of enterocyte-specific biomarkers.

In an embodiment, the stool-derived eukaryotic RNA biomarkers comprise inflammatory or inflammatory-related biomarkers, for instance lymphocyte-specific biomarkers. In an embodiment, the stool-derived eukaryotic RNA biomarkers consist essentially of lymphocyte-specific biomarkers. In an embodiment, the stool-derived eukaryotic RNA biomarkers consist of lymphocyte-specific biomarkers.

In an embodiment, the stool-derived eukaryotic RNA biomarkers comprise enterocyte-specific biomarkers and lymphocyte-specific biomarkers. In an embodiment, the stool-derived eukaryotic RNA biomarkers consist essentially of enterocyte-specific biomarkers and lymphocyte-specific biomarkers. In an embodiment, the stool-derived eukaryotic RNA biomarkers consist of enterocyte-specific biomarkers and lymphocyte-specific biomarkers.

In an embodiment, the stool-derived eukaryotic RNA biomarkers comprise one or more non-therapeutic targets. In an embodiment, the one or more non-therapeutic targets are selected from the group consisting of RNA transcripts that mediate cellular cytoskeleton remodeling, growth, and inflammatory response.

In an embodiment, the stool-derived eukaryotic RNA biomarkers comprise one or more therapeutic targets. In an embodiment, the one or more therapeutic targets are selected from the group consisting of TNF, MADCAM1, ITGA4, JAK1, TYK2, IL-12, IL-23, and any combination thereof. In an embodiment, the therapeutic target is TNF. In an embodiment, the therapeutic target is MADCAM1. In an embodiment, the therapeutic target is ITGA4. In an embodiment, the therapeutic target is JAK1. In an embodiment, the therapeutic target is TYK2. In an embodiment, the therapeutic target is IL-12. In an embodiment, the therapeutic target is IL-23.

In an embodiment, the one or more therapeutic targets are selected from the group consisting of a4B7, CCR6, CD30 ligand, FPR1, GLP-2, IL-2, IL-4/IL-13, IL7R, IL-10, IL-12/IL-23, IL-36, JAK, JAK1, JAK (ITK/TXK/JAK3), JAK3/TEC, MC1r, MRP2, NLRP3 inflammasome, NLRX1, PDE4, PSGL-1, RIPK1, SIP1, TLIA, TLR9, TNFa, TNFSF15, TPL2, TREM1, TYK2, and any combination thereof. In an embodiment, the therapeutic target is a4B7. In an embodiment, the therapeutic target is CCR6. In an embodiment, the therapeutic target is CD30 ligand. In an embodiment, the therapeutic target is FPR1. In an embodiment, the therapeutic target is GLP-2. In an embodiment, the therapeutic target is IL-2. In an embodiment, the therapeutic target is IL-4/IL-13. In an embodiment, the therapeutic target is IL7R. In an embodiment, the therapeutic target is IL-10. In an embodiment, the therapeutic target is IL-12/IL-23. In an embodiment, the therapeutic target is IL-36. In an embodiment, the therapeutic target is JAK. In an embodiment, the therapeutic target is JAK1. In an embodiment, the therapeutic target is JAK (ITK/TXK/JAK3). In an embodiment, the therapeutic target is JAK3/TEC. In an embodiment, the therapeutic target is MC1r. In an embodiment, the therapeutic target is MRP2. In an embodiment, the therapeutic target is NLRP3 inflammasome. In an embodiment, the therapeutic target is NLRX1. In an embodiment, the therapeutic target is PDE4. In an embodiment, the therapeutic target is PSGL-1. In an embodiment, the therapeutic target is RIPK1. In an embodiment, the therapeutic target is S1P1. In an embodiment, the therapeutic target is TLIA. In an embodiment, the therapeutic target is TLR9. In an embodiment, the therapeutic target is TNFa. In an embodiment, the therapeutic target is TNFSF15. In an embodiment, the therapeutic target is TPL2. In an embodiment, the therapeutic target is TREM1. In an embodiment, the therapeutic target is TYK2.

In an embodiment, the assessment comprises monitoring of mucosal lesions in the subject. In an embodiment, the assessment comprises evaluation of inflammation in the subject. In an embodiment, the evaluation of inflammation comprises analysis of quantity of inflammation. In an embodiment, the evaluation of inflammation comprises analysis of an increase or a decrease in inflammation. In an embodiment, the evaluation of inflammation comprises analysis of change in inflammation.

In an embodiment, the assessment comprises evaluation of a cell type in the subject. In an embodiment, the evaluation comprises analysis of quantity of the cell type. In an embodiment, the evaluation comprises analysis of an increase or a decrease of the cell type. In an embodiment, the evaluation comprises analysis of a change in relative proportion of the cell type. In an embodiment, the cell type is an inflammatory cell. In an embodiment, the cell type is an immunogenic cell. In an embodiment, the cell type is an enterocyte cell. In an embodiment, the cell type is an enterocyte-related cell. In an embodiment, the cell type is a lymphocyte cell. In an embodiment, the cell type is a lymphocyte-related cell. In an embodiment, the cell type is a T cell. In an embodiment, the cell type is an NK cell. In an embodiment, the cell type is a B cell. In an embodiment, the cell type is a macrophage. In an embodiment, the cell type is a neutrophil. In an embodiment, the cell type is an endothelial cell. In an embodiment, the cell type is a fibroblast.

In an embodiment, the assessment is a prognosis of therapeutic response to a medical intervention in the subject. In an embodiment, the medical intervention is a drug therapy. In an embodiment, the drug therapy is an IBD drug therapy. In an embodiment, the drug therapy is a UC drug therapy. In an embodiment, the drug therapy is a CD drug therapy. In an embodiment, the drug therapy comprises an anti-inflammatory drug. In an embodiment, the drug therapy comprises an immune system suppressing drug. In an embodiment, the drug therapy comprises a biologic. In an embodiment, the drug therapy comprises an antibiotic. The drug therapy can include, for example, adalimumab (Humira) or ustekinumab (Stelara) or vedolizumab (Entyio) or infliximab (Remicade) or tofacitinib (Xeljanz) or certolizumab (Cimzia) or golimumab (Simponi) or natalizumab (Tysabri) or ozanimod (Zeposia) or risankizumab-rzaa (Skyrizi) or upadacitinib (Rinvoq) or etrasimod (Velsipity) or mirikizumab or guselkumab or filgotinib. In an embodiment, the medical intervention is a colonoscopy.

In an embodiment, the assessment is a determination of therapeutic effectiveness of a medical intervention in the subject. In an embodiment, the medical intervention is a drug therapy. In an embodiment, the drug therapy is an IBD drug therapy. In an embodiment, the drug therapy is a UC drug therapy. In an embodiment, the drug therapy is a CD drug therapy. In an embodiment, the drug therapy comprises an anti-inflammatory drug. In an embodiment, the drug therapy comprises an immune system suppressing drug. In an embodiment, the drug therapy comprises a biologic. In an embodiment, the drug therapy comprises an antibiotic. In an embodiment, the medical intervention is a colonoscopy. The drug therapy can include, for example, adalimumab (Humira) or ustekinumab (Stelara) or vedolizumab (Entyio) or infliximab (Remicade) or tofacitinib (Xeljanz) or certolizumab (Cimzia) or golimumab (Simponi) or natalizumab (Tysabri) or ozanimod (Zeposia) or risankizumab-rzaa (Skyrizi) or upadacitinib (Rinvoq) or etrasimod (Velsipity) or mirikizumab or guselkumab or filgotinib.

In an embodiment, measuring the level of expression of nucleic acid biomarkers comprises nucleic acid extraction from the eukaryotic cells. In an embodiment, measuring the level of expression of nucleic acid biomarkers comprises DNA extraction from the eukaryotic cells. In an embodiment, measuring the level of expression of nucleic acid biomarkers comprises RNA extraction from the eukaryotic cells. In an embodiment, measuring the level of expression of nucleic acid biomarkers comprises next generation sequencing.

In an embodiment, the method further comprises diagnosing disease in the subject. In an embodiment, the disease is inflammatory bowel disease (IBD). In an embodiment, the disease is ulcerative colitis (UC). In an embodiment, the disease is Crohn's disease (CD).

In an embodiment, the method further comprises treating disease in the subject. In an embodiment, the disease is inflammatory bowel disease (IBD). In an embodiment, the disease is ulcerative colitis (UC). In an embodiment, the disease is Crohn's disease (CD).

In an embodiment, treating disease comprises administering an IBD drug therapy to the subject. In an embodiment, treating disease comprises administering a UC drug therapy to the subject. In an embodiment, treating disease comprises administering a CD drug therapy to the subject. In an embodiment, treating disease comprises administering an anti-inflammatory drug to the subject. In an embodiment, treating disease comprises administering an immune system suppressing drug to the subject. In an embodiment, treating disease comprises administering a biologic to the subject. In an embodiment, treating disease comprises administering an antibiotic to the subject. The immune system suppressing drug can include, for example, adalimumab (Humira) or ustekinumab (Stelara) or vedolizumab (Entyio) or infliximab (Remicade) or tofacitinib (Xeljanz) or certolizumab (Cimzia) or golimumab (Simponi) or natalizumab (Tysabri) or ozanimod (Zeposia) or risankizumab-rzaa (Skyrizi) or upadacitinib (Rinvoq) or etrasimod (Velsipity) or mirikizumab or guselkumab or filgotinib.

In an illustrative aspect, a method of predicting therapeutic response to a disease in a subject is provided. The method comprises the steps of measuring the level of expression of one or more stool-derived eukaryotic nucleic acid biomarkers selected from the biomarkers described herein in eukaryotic nucleic acid extracted from a stool sample from the subject, wherein the measured expression level of the one or more stool-derived eukaryotic nucleic acid biomarkers in the stool sample indicates therapeutic response to the disease in the subject.

In an embodiment, the method comprises comparing the measured expression level of the one or more stool-derived eukaryotic nucleic acid biomarkers in the stool sample with the measured expression level of the one or more stool-derived eukaryotic nucleic acid biomarkers in a control, wherein a difference in the measured expression level of the one or more stool-derived eukaryotic nucleic acid biomarkers in the stool sample relative to the measured expression level of the one or more stool-derived eukaryotic nucleic acid biomarkers in the control indicates therapeutic response to the disease in the subject.

In an embodiment, the nucleic acid is DNA. In an embodiment, the nucleic acid is RNA. In an embodiment, the nucleic acid is a combination of DNA and RNA. In an embodiment, the disease is inflammatory bowel disease (IBD). In an embodiment, the disease is ulcerative colitis (UC). In an embodiment, the disease is Crohn's disease (CD).

In an embodiment, the therapeutic response comprises an assessment of disease activity in the subject. In an embodiment, the assessment is a determination of active IBD symptoms in the subject. In an embodiment, the determination of active IBD symptoms indicates mild IBD symptoms. In an embodiment, the determination of active IBD symptoms indicates moderate IBD symptoms. In an embodiment, the determination of active IBD symptoms indicates severe IBD symptoms. In an embodiment, the assessment is a determination of IBD remission in the subject.

In an embodiment, the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 1. In an embodiment, the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 2. In an embodiment, the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 3. In an embodiment, the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 4. In an embodiment, the stool-derived eukaryotic nucleic acid biomarkers comprise a combination of biomarkers listed in Table 5.

In an embodiment, the one or more stool-derived eukaryotic nucleic acid biomarkers comprise stool-derived eukaryotic RNA biomarkers. In an embodiment, the stool-derived eukaryotic RNA biomarkers comprise enterocyte-specific biomarkers. In an embodiment, the stool-derived eukaryotic RNA biomarkers consist essentially of enterocyte-specific biomarkers. In an embodiment, the stool-derived eukaryotic RNA biomarkers consist of enterocyte-specific biomarkers.

In an embodiment, the stool-derived eukaryotic RNA biomarkers comprise lymphocyte-specific biomarkers. In an embodiment, the stool-derived eukaryotic RNA biomarkers consist essentially of lymphocyte-specific biomarkers. In an embodiment, the stool-derived eukaryotic RNA biomarkers consist of lymphocyte-specific biomarkers.

In an embodiment, the stool-derived eukaryotic RNA biomarkers comprise enterocyte-specific biomarkers and lymphocyte-specific biomarkers. In an embodiment, the stool-derived eukaryotic RNA biomarkers consist essentially of enterocyte-specific biomarkers and lymphocyte-specific biomarkers. In an embodiment, the stool-derived eukaryotic RNA biomarkers consist of enterocyte-specific biomarkers and lymphocyte-specific biomarkers.

In an embodiment, the stool-derived eukaryotic RNA biomarkers comprise one or more non-therapeutic targets. In an embodiment, the one or more non-therapeutic targets are selected from the group consisting of RNA transcripts that mediate cellular cytoskeleton remodeling, growth, and inflammatory response.

In an embodiment, the stool-derived eukaryotic RNA biomarkers comprise one or more therapeutic targets. In an embodiment, the one or more therapeutic targets are selected from the group consisting of a4B7, CCR6, CD30 ligand, FPR1, GLP-2, IL-2, IL-4/IL-13, IL7R, IL-10, IL-12/IL-23, IL-36, JAK, JAK1, JAK (ITK/TXK/JAK3), JAK3/TEC, MC1r, MRP2, NLRP3 inflammasome, NLRX1, PDE4, PSGL-1, RIPK1, S1P1, TL1A, TLR9, TNFa, TNFSF15, TPL2, TREM1, TYK2, and any combination thereof. In an embodiment, the therapeutic target is a4B7. In an embodiment, the therapeutic target is CCR6. In an embodiment, the therapeutic target is CD30 ligand. In an embodiment, the therapeutic target is FPR1. In an embodiment, the therapeutic target is GLP-2. In an embodiment, the therapeutic target is IL-2. In an embodiment, the therapeutic target is IL-4/IL-13. In an embodiment, the therapeutic target is IL7R. In an embodiment, the therapeutic target is IL-10. In an embodiment, the therapeutic target is IL-12/IL-23. In an embodiment, the therapeutic target is IL-36. In an embodiment, the therapeutic target is JAK. In an embodiment, the therapeutic target is JAK1. In an embodiment, the therapeutic target is JAK (ITK/TXK/JAK3). In an embodiment, the therapeutic target is JAK3/TEC. In an embodiment, the therapeutic target is MC1r. In an embodiment, the therapeutic target is MRP2. In an embodiment, the therapeutic target is NLRP3 inflammasome. In an embodiment, the therapeutic target is NLRX1. In an embodiment, the therapeutic target is PDE4. In an embodiment, the therapeutic target is PSGL-1. In an embodiment, the therapeutic target is RIPK1. In an embodiment, the therapeutic target is S1P1. In an embodiment, the therapeutic target is TLIA. In an embodiment, the therapeutic target is TLR9. In an embodiment, the therapeutic target is TNFa. In an embodiment, the therapeutic target is TNFSF15. In an embodiment, the therapeutic target is TPL2. In an embodiment, the therapeutic target is TREM1. In an embodiment, the therapeutic target is TYK2.

In an embodiment, the assessment comprises monitoring of mucosal lesions in the subject. In an embodiment, the assessment comprises evaluation of inflammation in the subject. In an embodiment, the evaluation of inflammation comprises analysis of quantity of inflammation. In an embodiment, the evaluation of inflammation comprises analysis of an increase or a decrease in inflammation. In an embodiment, the evaluation of inflammation comprises analysis of change in inflammation.

In an embodiment, the assessment comprises evaluation of a cell type in the subject. In an embodiment, the evaluation comprises analysis of quantity of the cell type. In an embodiment, the evaluation comprises analysis of an increase or a decrease of the cell type. In an embodiment, the evaluation comprises analysis of a change in relative proportion of the cell type. In an embodiment, the cell type is an inflammatory cell. In an embodiment, the cell type is an immunogenic cell. In an embodiment, the cell type is an enterocyte cell. In an embodiment, the cell type is an enterocyte-related cell. In an embodiment, the cell type is a lymphocyte cell. In an embodiment, the cell type is a lymphocyte-related cell. In an embodiment, the cell type is a T cell. In an embodiment, the cell type is an NK cell. In an embodiment, the cell type is a B cell. In an embodiment, the cell type is a macrophage. In an embodiment, the cell type is a neutrophil. In an embodiment, the cell type is an endothelial cell. In an embodiment, the cell type is a fibroblast.

In an embodiment, the therapeutic response is to a medical intervention in the subject. In an embodiment, the medical intervention is a drug therapy. In an embodiment, the drug therapy is an IBD drug therapy. In an embodiment, the drug therapy is a UC drug therapy. In an embodiment, the drug therapy is a CD drug therapy. In an embodiment, the drug therapy comprises an anti-inflammatory drug. In an embodiment, the drug therapy comprises an immune system suppressing drug. In an embodiment, the drug therapy comprises a biologic. In an embodiment, the drug therapy comprises an antibiotic. The drug therapy can include, for example, adalimumab (Humira) or ustekinumab (Stelara) or vedolizumab (Entyio) or infliximab (Remicade) or tofacitinib (Xeljanz) or certolizumab (Cimzia) or golimumab (Simponi) or natalizumab (Tysabri) or ozanimod (Zeposia) or risankizumab-rzaa (Skyrizi) or upadacitinib (Rinvoq) or etrasimod (Velsipity) or mirikizumab or guselkumab or filgotinib. In an embodiment, the medical intervention is a colonoscopy.

In an embodiment, measuring the level of expression of nucleic acid biomarkers comprises nucleic acid extraction from the eukaryotic cells. In an embodiment, measuring the level of expression of nucleic acid biomarkers comprises DNA extraction from the eukaryotic cells. In an embodiment, measuring the level of expression of nucleic acid biomarkers comprises RNA extraction from the eukaryotic cells. In an embodiment, measuring the level of expression of nucleic acid biomarkers comprises next generation sequencing.

In an embodiment, the method further comprises c) treating disease in the subject. In an embodiment, the disease is inflammatory bowel disease (IBD). In an embodiment, the disease is ulcerative colitis (UC). In an embodiment, the disease is Crohn's disease (CD).

In an embodiment, treating disease comprises administering an IBD drug therapy to the subject. In an embodiment, treating disease comprises administering a UC drug therapy to the subject. In an embodiment, treating disease comprises administering a CD drug therapy to the subject. In an embodiment, treating disease comprises administering an anti-inflammatory drug to the subject. In an embodiment, treating disease comprises administering an immune system suppressing drug to the subject. In an embodiment, treating disease comprises administering a biologic to the subject. In an embodiment, treating disease comprises administering an antibiotic to the subject. The immune system suppressing drug can include, for example, adalimumab (Humira) or ustekinumab (Stelara) or vedolizumab (Entyio) or infliximab (Remicade) or tofacitinib (Xeljanz) or certolizumab (Cimzia) or golimumab (Simponi) or natalizumab (Tysabri) or ozanimod (Zeposia) or risankizumab-rzaa (Skyrizi) or upadacitinib (Rinvoq) or etrasimod (Velsipity) or mirikizumab or guselkumab or filgotinib.

In an illustrative aspect, a method of assessing the presence and/or the mechanism of inflammation in a subject is provided. The method comprises the steps of measuring the level of expression of one or more stool-derived eukaryotic nucleic acid biomarkers selected from the biomarkers described herein in eukaryotic nucleic acid extracted from a stool sample from the subject, wherein the measured expression level of the one or more stool-derived eukaryotic nucleic acid biomarkers in the stool sample indicates the assessment of inflammation in the subject.

In an embodiment, the method comprises comparing the measured expression level of the one or more stool-derived eukaryotic nucleic acid biomarkers in the stool sample with the measured expression level of the one or more stool-derived eukaryotic nucleic acid biomarkers in a control, wherein a difference in the measured expression level of the one or more stool-derived eukaryotic nucleic acid biomarkers in the stool sample relative to the measured expression level of the one or more stool-derived eukaryotic nucleic acid biomarkers in the control indicates the assessment of inflammation in the subject.

In an embodiment, the nucleic acid is DNA. In an embodiment, the nucleic acid is RNA. In an embodiment, the nucleic acid is a combination of DNA and RNA.

In an embodiment, the inflammation is associated with a disease. In an embodiment, the disease is inflammatory bowel disease (IBD). In an embodiment, the disease is ulcerative colitis (UC). In an embodiment, the disease is Crohn's disease (CD).

In an embodiment, the assessment of inflammation comprises an assessment of disease activity in the subject. In an embodiment, the assessment is a determination of active IBD symptoms in the subject. In an embodiment, the determination of active IBD symptoms indicates mild IBD symptoms. In an embodiment, the determination of active IBD symptoms indicates moderate IBD symptoms. In an embodiment, the determination of active IBD symptoms indicates severe IBD symptoms. In an embodiment, the assessment is a determination of IBD remission in the subject.

In an embodiment, the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 1. In an embodiment, the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 2. In an embodiment, the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 3. In an embodiment, the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 4. In an embodiment, the stool-derived eukaryotic nucleic acid biomarkers comprise a combination of biomarkers listed in Table 5.

In an embodiment, the one or more stool-derived eukaryotic nucleic acid biomarkers comprise stool-derived eukaryotic RNA biomarkers. In an embodiment, the stool-derived eukaryotic RNA biomarkers comprise enterocyte-specific biomarkers. In an embodiment, the stool-derived eukaryotic RNA biomarkers consist essentially of enterocyte-specific biomarkers. In an embodiment, the stool-derived eukaryotic RNA biomarkers consist of enterocyte-specific biomarkers.

In an embodiment, the stool-derived eukaryotic RNA biomarkers comprise lymphocyte-specific biomarkers. In an embodiment, the stool-derived eukaryotic RNA biomarkers consist essentially of lymphocyte-specific biomarkers. In an embodiment, the stool-derived eukaryotic RNA biomarkers consist of lymphocyte-specific biomarkers.

In an embodiment, the stool-derived eukaryotic RNA biomarkers comprise enterocyte-specific biomarkers and lymphocyte-specific biomarkers. In an embodiment, the stool-derived eukaryotic RNA biomarkers consist essentially of enterocyte-specific biomarkers and lymphocyte-specific biomarkers. In an embodiment, the stool-derived eukaryotic RNA biomarkers consist of enterocyte-specific biomarkers and lymphocyte-specific biomarkers.

In an embodiment, the stool-derived eukaryotic RNA biomarkers comprise one or more non-therapeutic targets. In an embodiment, the one or more non-therapeutic targets are selected from the group consisting of RNA transcripts that mediate cellular cytoskeleton remodeling, growth, and inflammatory response.

In an embodiment, the stool-derived eukaryotic RNA biomarkers comprise one or more therapeutic targets. In an embodiment, the one or more therapeutic targets are selected from the group consisting of a4B7, CCR6, CD30 ligand, FPR1, GLP-2, IL-2, IL-4/IL-13, IL7R, IL-10, IL-12/IL-23, IL-36, JAK, JAK1, JAK (ITK/TXK/JAK3), JAK3/TEC, MC1r, MRP2, NLRP3 inflammasome, NLRX1, PDE4, PSGL-1, RIPK1, S1P1, TLIA, TLR9, TNFa, TNFSF15, TPL2, TREM1, TYK2, and any combination thereof. In an embodiment, the therapeutic target is a4B7. In an embodiment, the therapeutic target is CCR6. In an embodiment, the therapeutic target is CD30 ligand. In an embodiment, the therapeutic target is FPR1. In an embodiment, the therapeutic target is GLP-2. In an embodiment, the therapeutic target is IL-2. In an embodiment, the therapeutic target is IL-4/IL-13. In an embodiment, the therapeutic target is IL7R. In an embodiment, the therapeutic target is IL-10. In an embodiment, the therapeutic target is IL-12/IL-23. In an embodiment, the therapeutic target is IL-36. In an embodiment, the therapeutic target is JAK. In an embodiment, the therapeutic target is JAK1. In an embodiment, the therapeutic target is JAK (ITK/TXK/JAK3). In an embodiment, the therapeutic target is JAK3/TEC. In an embodiment, the therapeutic target is MC1r. In an embodiment, the therapeutic target is MRP2. In an embodiment, the therapeutic target is NLRP3 inflammasome. In an embodiment, the therapeutic target is NLRX1. In an embodiment, the therapeutic target is PDE4. In an embodiment, the therapeutic target is PSGL-1. In an embodiment, the therapeutic target is RIPK1. In an embodiment, the therapeutic target is S1P1. In an embodiment, the therapeutic target is TL1A. In an embodiment, the therapeutic target is TLR9. In an embodiment, the therapeutic target is TNFa. In an embodiment, the therapeutic target is TNFSF15. In an embodiment, the therapeutic target is TPL2. In an embodiment, the therapeutic target is TREM1. In an embodiment, the therapeutic target is TYK2.

In an embodiment, the assessment comprises monitoring of mucosal lesions in the subject. In an embodiment, the assessment comprises evaluation of inflammation in the subject. In an embodiment, the evaluation of inflammation comprises analysis of quantity of inflammation. In an embodiment, the evaluation of inflammation comprises analysis of an increase or a decrease in inflammation. In an embodiment, the evaluation of inflammation comprises analysis of change in inflammation.

In an embodiment, the assessment comprises evaluation of a cell type in the subject. In an embodiment, the evaluation comprises analysis of quantity of the cell type. In an embodiment, the evaluation comprises analysis of an increase or a decrease of the cell type. In an embodiment, the evaluation comprises analysis of a change in relative proportion of the cell type. In an embodiment, the cell type is an inflammatory cell. In an embodiment, the cell type is an immunogenic cell. In an embodiment, the cell type is an enterocyte cell. In an embodiment, the cell type is an enterocyte-related cell. In an embodiment, the cell type is a lymphocyte cell. In an embodiment, the cell type is a lymphocyte-related cell. In an embodiment, the cell type is a T cell. In an embodiment, the cell type is an NK cell. In an embodiment, the cell type is a B cell. In an embodiment, the cell type is a macrophage. In an embodiment, the cell type is a neutrophil. In an embodiment, the cell type is an endothelial cell. In an embodiment, the cell type is a fibroblast.

In an embodiment, the assessment comprises a prognosis of therapeutic response to a medical intervention in the subject. In an embodiment, the medical intervention is a drug therapy. In an embodiment, the drug therapy is an IBD drug therapy. In an embodiment, the drug therapy is a UC drug therapy. In an embodiment, the drug therapy is a CD drug therapy. In an embodiment, the drug therapy comprises an anti-inflammatory drug. In an embodiment, the drug therapy comprises an immune system suppressing drug. In an embodiment, the drug therapy comprises a biologic. In an embodiment, the drug therapy comprises an antibiotic. The drug therapy can include, for example, adalimumab (Humira) or ustekinumab (Stelara) or vedolizumab (Entyio) or infliximab (Remicade) or tofacitinib (Xeljanz) or certolizumab (Cimzia) or golimumab (Simponi) or natalizumab (Tysabri) or ozanimod (Zeposia) or risankizumab-rzaa (Skyrizi) or upadacitinib (Rinvoq) or etrasimod (Velsipity) or mirikizumab or guselkumab or filgotinib. In an embodiment, the medical intervention is a colonoscopy.

In an embodiment, the assessment comprises a determination of therapeutic effectiveness of a medical intervention in the subject. In an embodiment, the medical intervention is a drug therapy. In an embodiment, the drug therapy is an IBD drug therapy. In an embodiment, the drug therapy is a UC drug therapy. In an embodiment, the drug therapy is a CD drug therapy. In an embodiment, the drug therapy comprises an anti-inflammatory drug. In an embodiment, the drug therapy comprises an immune system suppressing drug. In an embodiment, the drug therapy comprises a biologic. In an embodiment, the drug therapy comprises an antibiotic. The drug therapy can include, for example, adalimumab (Humira) or ustekinumab (Stelara) or vedolizumab (Entyio) or infliximab (Remicade) or tofacitinib (Xeljanz) or certolizumab (Cimzia) or golimumab (Simponi) or natalizumab (Tysabri) or ozanimod (Zeposia) or risankizumab-rzaa (Skyrizi) or upadacitinib (Rinvoq) or etrasimod (Velsipity) or mirikizumab or guselkumab or filgotinib. In an embodiment, the medical intervention is a colonoscopy.

In an embodiment, measuring the level of expression of nucleic acid biomarkers comprises nucleic acid extraction from the eukaryotic cells. In an embodiment, measuring the level of expression of nucleic acid biomarkers comprises DNA extraction from the eukaryotic cells. In an embodiment, measuring the level of expression of nucleic acid biomarkers comprises RNA extraction from the eukaryotic cells. In an embodiment, measuring the level of expression of nucleic acid biomarkers comprises next generation sequencing.

In an embodiment, the method further comprises c) diagnosing disease in the subject. In an embodiment, the disease is inflammatory bowel disease (IBD). In an embodiment, the disease is ulcerative colitis (UC). In an embodiment, the disease is Crohn's disease (CD).

In an embodiment, the method further comprises c) treating disease in the subject. In an embodiment, the disease is inflammatory bowel disease (IBD). In an embodiment, the disease is ulcerative colitis (UC). In an embodiment, the disease is Crohn's disease (CD). In an embodiment, treating disease comprises administering an IBD drug therapy to the subject. In an embodiment, treating disease comprises administering a UC drug therapy to the subject. In an embodiment, treating disease comprises administering a CD drug therapy to the subject. In an embodiment, treating disease comprises administering an anti-inflammatory drug to the subject. In an embodiment, treating disease comprises administering an immune system suppressing drug to the subject. In an embodiment, treating disease comprises administering a biologic to the subject. In an embodiment, treating disease comprises administering an antibiotic to the subject. The immune system suppressing drug can include, for example, adalimumab (Humira) or ustekinumab (Stelara) or vedolizumab (Entyio) or infliximab (Remicade) or tofacitinib (Xeljanz) or certolizumab (Cimzia) or golimumab (Simponi) or natalizumab (Tysabri) or ozanimod (Zeposia) or risankizumab-rzaa (Skyrizi) or upadacitinib (Rinvoq) or etrasimod (Velsipity) or mirikizumab or guselkumab or filgotinib.

In an illustrative aspect, a method of assessing one or more immune cells or immune-related cells in a subject is provided. The method comprises the steps of measuring the level of expression of one or more stool-derived eukaryotic nucleic acid biomarkers selected from the biomarkers described herein in eukaryotic nucleic acid extracted from a stool sample from the subject; wherein the measured expression level of the one or more stool-derived eukaryotic nucleic acid biomarkers in the stool sample indicates the assessment of the one or more immune cells in the subject. For instance, immune-related cells can include fibroblasts, stromal cells, and the like.

In an embodiment, the method comprises comparing the measured expression level of the one or more stool-derived eukaryotic nucleic acid biomarkers in the stool sample with the measured expression level of the one or more stool-derived eukaryotic nucleic acid biomarkers in a control, wherein a difference in the measured expression level of the one or more stool-derived eukaryotic nucleic acid biomarkers in the stool sample relative to the measured expression level of the one or more stool-derived eukaryotic nucleic acid biomarkers in the control indicates the assessment of the immune cell in the subject.

In an embodiment, the nucleic acid is DNA. In an embodiment, the nucleic acid is RNA. In an embodiment, the nucleic acid is a combination of DNA and RNA.

In an embodiment, the immune cell is associated with a disease. In an embodiment, the disease is inflammatory bowel disease (IBD). In an embodiment, the disease is ulcerative colitis (UC). In an embodiment, the disease is Crohn's disease (CD).

In an embodiment, the assessment of immune cells comprises an assessment of disease activity in the subject. In an embodiment, the assessment is a determination of active IBD symptoms in the subject. In an embodiment, the determination of active IBD symptoms indicates mild IBD symptoms. In an embodiment, the determination of active IBD symptoms indicates moderate IBD symptoms. In an embodiment, the determination of active IBD symptoms indicates severe IBD symptoms. In an embodiment, the assessment is a determination of IBD remission in the subject.

In an embodiment, the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 1. In an embodiment, the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 2. In an embodiment, the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 3. In an embodiment, the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 4. In an embodiment, the stool-derived eukaryotic nucleic acid biomarkers comprise a combination of biomarkers listed in Table 5.

In an embodiment, the one or more stool-derived eukaryotic nucleic acid biomarkers comprise stool-derived eukaryotic RNA biomarkers. In an embodiment, the stool-derived eukaryotic RNA biomarkers comprise enterocyte-specific biomarkers. In an embodiment, the stool-derived eukaryotic RNA biomarkers consist essentially of enterocyte-specific biomarkers. In an embodiment, the stool-derived eukaryotic RNA biomarkers consist of enterocyte-specific biomarkers.

In an embodiment, the stool-derived eukaryotic RNA biomarkers comprise lymphocyte-specific biomarkers. In an embodiment, the stool-derived eukaryotic RNA biomarkers consist essentially of lymphocyte-specific biomarkers. In an embodiment, the stool-derived eukaryotic RNA biomarkers consist of lymphocyte-specific biomarkers.

In an embodiment, the stool-derived eukaryotic RNA biomarkers comprise enterocyte-specific biomarkers and lymphocyte-specific biomarkers. In an embodiment, the stool-derived eukaryotic RNA biomarkers consist essentially of enterocyte-specific biomarkers and lymphocyte-specific biomarkers. In an embodiment, the stool-derived eukaryotic RNA biomarkers consist of enterocyte-specific biomarkers and lymphocyte-specific biomarkers.

In an embodiment, the stool-derived eukaryotic RNA biomarkers comprise one or more non-therapeutic targets. In an embodiment, the one or more non-therapeutic targets are selected from the group consisting of RNA transcripts that mediate cellular cytoskeleton remodeling, growth, and inflammatory response.

In an embodiment, the stool-derived eukaryotic RNA biomarkers comprise one or more therapeutic targets. In an embodiment, the one or more therapeutic targets are selected from the group consisting of a4B7, CCR6, CD30 ligand, FPR1, GLP-2, IL-2, IL-4/IL-13, IL7R, IL-10, IL-12/IL-23, IL-36, JAK, JAK1, JAK (ITK/TXK/JAK3), JAK3/TEC, MC1r, MRP2, NLRP3 inflammasome, NLRX1, PDE4, PSGL-1, RIPK1, S1P1, TL1A, TLR9, TNFa, TNFSF15, TPL2, TREM1, TYK2, and any combination thereof. In an embodiment, the therapeutic target is a4B7. In an embodiment, the therapeutic target is CCR6. In an embodiment, the therapeutic target is CD30 ligand. In an embodiment, the therapeutic target is FPR1. In an embodiment, the therapeutic target is GLP-2. In an embodiment, the therapeutic target is IL-2. In an embodiment, the therapeutic target is IL-4/IL-13. In an embodiment, the therapeutic target is IL7R. In an embodiment, the therapeutic target is IL-10. In an embodiment, the therapeutic target is IL-12/IL-23. In an embodiment, the therapeutic target is IL-36. In an embodiment, the therapeutic target is JAK. In an embodiment, the therapeutic target is JAK1. In an embodiment, the therapeutic target is JAK (ITK/TXK/JAK3). In an embodiment, the therapeutic target is JAK3/TEC. In an embodiment, the therapeutic target is MC1r. In an embodiment, the therapeutic target is MRP2. In an embodiment, the therapeutic target is NLRP3 inflammasome. In an embodiment, the therapeutic target is NLRX1. In an embodiment, the therapeutic target is PDE4. In an embodiment, the therapeutic target is PSGL-1. In an embodiment, the therapeutic target is RIPK1. In an embodiment, the therapeutic target is S1P1. In an embodiment, the therapeutic target is TL1A. In an embodiment, the therapeutic target is TLR9. In an embodiment, the therapeutic target is TNFa. In an embodiment, the therapeutic target is TNFSF15. In an embodiment, the therapeutic target is TPL2. In an embodiment, the therapeutic target is TREM1. In an embodiment, the therapeutic target is TYK2.

In an embodiment, the assessment comprises monitoring of mucosal lesions in the subject. In an embodiment, the assessment comprises evaluation of inflammation in the subject. In an embodiment, the evaluation of inflammation comprises analysis of quantity of inflammation. In an embodiment, the evaluation of inflammation comprises analysis of an increase or a decrease in inflammation. In an embodiment, the evaluation of inflammation comprises analysis of change in inflammation.

In an embodiment, the assessment comprises evaluation of a cell type in the subject. In an embodiment, the evaluation comprises analysis of quantity of the cell type. In an embodiment, the evaluation comprises analysis of an increase or a decrease of the cell type. In an embodiment, the evaluation comprises analysis of a change in relative proportion of the cell type. In an embodiment, the cell type is an inflammatory cell. In an embodiment, the cell type is an immunogenic cell. In an embodiment, the cell type is an enterocyte cell. In an embodiment, the cell type is an enterocyte-related cell. In an embodiment, the cell type is a lymphocyte cell. In an embodiment, the cell type is a lymphocyte-related cell. In an embodiment, the cell type is a T cell. In an embodiment, the cell type is an NK cell. In an embodiment, the cell type is a B cell. In an embodiment, the cell type is a macrophage. In an embodiment, the cell type is a neutrophil. In an embodiment, the cell type is an endothelial cell. In an embodiment, the cell type is a fibroblast.

In an embodiment, the assessment is a prognosis of therapeutic response to a medical intervention in the subject. In an embodiment, the medical intervention is a drug therapy. In an embodiment, the drug therapy is an IBD drug therapy. In an embodiment, the drug therapy is a UC drug therapy. In an embodiment, the drug therapy is a CD drug therapy. In an embodiment, the drug therapy comprises an anti-inflammatory drug. In an embodiment, the drug therapy comprises an immune system suppressing drug. In an embodiment, the drug therapy comprises a biologic. In an embodiment, the drug therapy comprises an antibiotic. In an embodiment, the medical intervention is a colonoscopy. The drug therapy can include, for example, adalimumab (Humira) or ustekinumab (Stelara) or vedolizumab (Entyio) or infliximab (Remicade) or tofacitinib (Xeljanz) or certolizumab (Cimzia) or golimumab (Simponi) or natalizumab (Tysabri) or ozanimod (Zeposia) or risankizumab-rzaa (Skyrizi) or upadacitinib (Rinvoq) or etrasimod (Velsipity) or mirikizumab or guselkumab or filgotinib.

In an embodiment, the assessment is a determination of therapeutic effectiveness of a medical intervention in the subject. In an embodiment, the medical intervention is a drug therapy. In an embodiment, the drug therapy is an IBD drug therapy. In an embodiment, the drug therapy is a UC drug therapy. In an embodiment, the drug therapy is a CD drug therapy. In an embodiment, the drug therapy comprises an anti-inflammatory drug. In an embodiment, the drug therapy comprises an immune system suppressing drug. In an embodiment, the drug therapy comprises a biologic. In an embodiment, the drug therapy comprises an antibiotic. In an embodiment, the medical intervention is a colonoscopy. The drug therapy can include, for example, adalimumab (Humira) or ustekinumab (Stelara) or vedolizumab (Entyio) or infliximab (Remicade) or tofacitinib (Xeljanz) or certolizumab (Cimzia) or golimumab (Simponi) or natalizumab (Tysabri) or ozanimod (Zeposia) or risankizumab-rzaa (Skyrizi) or upadacitinib (Rinvoq) or etrasimod (Velsipity) or mirikizumab or guselkumab or filgotinib.

In an embodiment, measuring the level of expression of nucleic acid biomarkers comprises nucleic acid extraction from the eukaryotic cells. In an embodiment, measuring the level of expression of nucleic acid biomarkers comprises DNA extraction from the eukaryotic cells. In an embodiment, measuring the level of expression of nucleic acid biomarkers comprises RNA extraction from the eukaryotic cells. In an embodiment, measuring the level of expression of nucleic acid biomarkers comprises next generation sequencing.

In an embodiment, the method further comprises c) diagnosing disease in the subject. In an embodiment, the disease is inflammatory bowel disease (IBD). In an embodiment, the disease is ulcerative colitis (UC). In an embodiment, the disease is Crohn's disease (CD).

In an embodiment, the method further comprises c) treating disease in the subject. In an embodiment, the disease is inflammatory bowel disease (IBD). In an embodiment, the disease is ulcerative colitis (UC). In an embodiment, the disease is Crohn's disease (CD). In an embodiment, treating disease comprises administering an IBD drug therapy to the subject. In an embodiment, treating disease comprises administering a UC drug therapy to the subject. In an embodiment, treating disease comprises administering a CD drug therapy to the subject. In an embodiment, treating disease comprises administering an anti-inflammatory drug to the subject. In an embodiment, treating disease comprises administering an immune system suppressing drug to the subject. In an embodiment, treating disease comprises administering a biologic to the subject. In an embodiment, treating disease comprises administering an antibiotic to the subject. The immune system suppressing drug can include, for example, adalimumab (Humira) or ustekinumab (Stelara) or vedolizumab (Entyio) or infliximab (Remicade) or tofacitinib (Xeljanz) or certolizumab (Cimzia) or golimumab (Simponi) or natalizumab (Tysabri) or ozanimod (Zeposia) or risankizumab-rzaa (Skyrizi) or upadacitinib (Rinvoq) or etrasimod (Velsipity) or mirikizumab or guselkumab or filgotinib.

The following numbered embodiments are contemplated and are non-limiting:
1. A method of assessing disease in a subject, the method comprising:
   measuring the level of expression of one or more stool-derived eukaryotic nucleic acid biomarkers selected from the biomarkers described herein in eukaryotic nucleic acid extracted from a stool sample from the subject,
   wherein the measured expression level of the one or more stool-derived eukaryotic nucleic acid biomarkers in the stool sample indicates the assessment of disease in the subject.
2. The method of clause 1, any other suitable clause, or any combination of suitable clauses, wherein the method comprises comparing the measured expression level of the one or more stool-derived eukaryotic nucleic acid biomarkers in the stool sample with the measured expression level of the one or more stool-derived eukaryotic nucleic acid biomarkers in a control, wherein a difference in the measured expression level of the one or more stool-derived eukaryotic nucleic acid biomarkers in the stool sample relative to the measured expression level of the one or more stool-derived eukaryotic nucleic acid biomarkers in the control indicates the assessment of disease in the subject.
3. The method of clause 1, any other suitable clause, or any combination of suitable clauses, wherein the nucleic acid is DNA.
4. The method of clause 1, any other suitable clause, or any combination of suitable clauses, wherein the nucleic acid is RNA.
5. The method of clause 1, any other suitable clause, or any combination of suitable clauses, wherein the nucleic acid is a combination of DNA and RNA.
6. The method of clause 1, any other suitable clause, or any combination of suitable clauses, wherein the disease is inflammatory bowel disease (IBD).
7. The method of clause 1, any other suitable clause, or any combination of suitable clauses, wherein the disease is ulcerative colitis (UC).
8. The method of clause 1, any other suitable clause, or any combination of suitable clauses, wherein the disease is Crohn's disease (CD).
9. The method of clause 1, any other suitable clause, or any combination of suitable clauses, wherein the assessment of disease comprises an assessment of disease activity in the subject.
10. The method of clause 9, any other suitable clause, or any combination of suitable clauses, wherein the assessment is a determination of active IBD symptoms in the subject.
11. The method of clause 10, any other suitable clause, or any combination of suitable clauses, wherein the determination of active IBD symptoms indicates mild IBD symptoms.
12. The method of clause 10, any other suitable clause, or any combination of suitable clauses, wherein the determination of active IBD symptoms indicates moderate IBD symptoms.
13. The method of clause 10, any other suitable clause, or any combination of suitable clauses, wherein the determination of active IBD symptoms indicates severe IBD symptoms.
14. The method of clause 9, any other suitable clause, or any combination of suitable clauses, wherein the assessment is a determination of IBD remission in the subject.
15. The method of clause 1, any other suitable clause, or any combination of suitable clauses, wherein the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 1.
16. The method of clause 1, any other suitable clause, or any combination of suitable clauses, wherein the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 2.
17. The method of clause 1, any other suitable clause, or any combination of suitable clauses, wherein the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 3.
18. The method of clause 1, any other suitable clause, or any combination of suitable clauses, wherein the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 4.
19. The method of clause 1, any other suitable clause, or any combination of suitable clauses, wherein the stool-derived eukaryotic nucleic acid biomarkers comprise a combination of biomarkers listed in Table 5.
20. The method of clause 1, any other suitable clause, or any combination of suitable clauses, wherein the one or more stool-derived eukaryotic nucleic acid biomarkers comprise stool-derived eukaryotic RNA biomarkers.
21. The method of clause 20, any other suitable clause, or any combination of suitable clauses, wherein the stool-derived eukaryotic RNA biomarkers comprise enterocyte-specific biomarkers.
22. The method of clause 20, any other suitable clause, or any combination of suitable clauses, wherein the stool-derived eukaryotic RNA biomarkers consist essentially of enterocyte-specific biomarkers.
23. The method of clause 20, any other suitable clause, or any combination of suitable clauses, wherein the stool-derived eukaryotic RNA biomarkers consist of enterocyte-specific biomarkers.
24. The method of clause 20, any other suitable clause, or any combination of suitable clauses, wherein the stool-derived eukaryotic RNA biomarkers comprise lymphocyte-specific biomarkers.
25. The method of clause 20, any other suitable clause, or any combination of suitable clauses, wherein the stool-derived eukaryotic RNA biomarkers consist essentially of lymphocyte-specific biomarkers.
26. The method of clause 20, any other suitable clause, or any combination of suitable clauses, wherein the stool-derived eukaryotic RNA biomarkers consist of lymphocyte-specific biomarkers.
27. The method of clause 20, any other suitable clause, or any combination of suitable clauses, wherein the stool-derived eukaryotic RNA biomarkers comprise enterocyte-specific biomarkers and lymphocyte-specific biomarkers.
28. The method of clause 20, any other suitable clause, or any combination of suitable clauses, wherein the stool-derived eukaryotic RNA biomarkers consist essentially of enterocyte-specific biomarkers and lymphocyte-specific biomarkers.
29. The method of clause 20, any other suitable clause, or any combination of suitable clauses, wherein the stool-derived eukaryotic RNA biomarkers consist of enterocyte-specific biomarkers and lymphocyte-specific biomarkers.
30. The method of clause 20, any other suitable clause, or any combination of suitable clauses, wherein the stool-derived eukaryotic RNA biomarkers comprise one or more non-therapeutic targets.
31. The method of clause 30, any other suitable clause, or any combination of suitable clauses, wherein the one or more non-therapeutic targets are selected from the group consisting of RNA transcripts that mediate cellular cytoskeleton remodeling, growth, and inflammatory response.
32. The method of clause 20, any other suitable clause, or any combination of suitable clauses, wherein the stool-derived eukaryotic RNA biomarkers comprise one or more therapeutic targets.
33. The method of clause 32, any other suitable clause, or any combination of suitable clauses, wherein the one or more therapeutic targets are selected from the group consisting of TNF, MADCAMI, ITGA4, JAK1, TYK2, IL-12, IL-23, and any combination thereof.
34. The method of clause 32, any other suitable clause, or any combination of suitable clauses, wherein the therapeutic target is TNF.
35. The method of clause 32, any other suitable clause, or any combination of suitable clauses, wherein the therapeutic target is MADCAM1.
36. The method of clause 32, any other suitable clause, or any combination of suitable clauses, wherein the therapeutic target is ITGA4.
37. The method of clause 32, any other suitable clause, or any combination of suitable clauses, wherein the therapeutic target is JAK1.
38. The method of clause 32, any other suitable clause, or any combination of suitable clauses, wherein the therapeutic target is TYK2.
39. The method of clause 32, any other suitable clause, or any combination of suitable clauses, wherein the therapeutic target is IL-12.
40. The method of clause 32, any other suitable clause, or any combination of suitable clauses, wherein the therapeutic target is IL-23.
41. The method of clause 32, any other suitable clause, or any combination of suitable clauses, wherein the one or more therapeutic targets are selected from the group consisting of a4B7, CCR6, CD30 ligand, FPR1, GLP-2, IL-2, IL-4/IL-13, IL7R, IL-10, IL-12/IL-23, IL-36, JAK, JAK1, JAK (ITK/TXK/JAK3), JAK3/TEC, MC1r, MRP2, NLRP3 inflammasome, NLRX1, PDE4, PSGL-1, RIPK1, S1P1, TL1A, TLR9, TNFa, TNFSF15, TPL2, TREM1, TYK2, and any combination thereof.
42. The method of clause 32, any other suitable clause, or any combination of suitable clauses, wherein the therapeutic target is a4B7.
43. The method of clause 32, any other suitable clause, or any combination of suitable clauses, wherein the therapeutic target is CCR6.
44. The method of clause 32, any other suitable clause, or any combination of suitable clauses, wherein the therapeutic target is CD30 ligand.
45. The method of clause 32, any other suitable clause, or any combination of suitable clauses, wherein the therapeutic target is FPR1.
46. The method of clause 32, any other suitable clause, or any combination of suitable clauses, wherein the therapeutic target is GLP-2.
47. The method of clause 32, any other suitable clause, or any combination of suitable clauses, wherein the therapeutic target is IL-2.
48. The method of clause 32, any other suitable clause, or any combination of suitable clauses, wherein the therapeutic target is IL-4/IL-13.
49. The method of clause 32, any other suitable clause, or any combination of suitable clauses, wherein the therapeutic target is IL7R.
50. The method of clause 32, any other suitable clause, or any combination of suitable clauses, wherein the therapeutic target is IL-10.
51. The method of clause 32, any other suitable clause, or any combination of suitable clauses, wherein the therapeutic target is IL-12/IL-23.
52. The method of clause 32, any other suitable clause, or any combination of suitable clauses, wherein the therapeutic target is IL-36.
53. The method of clause 32, any other suitable clause, or any combination of suitable clauses, wherein the therapeutic target is JAK.
54. The method of clause 32, any other suitable clause, or any combination of suitable clauses, wherein the therapeutic target is JAK1.
55. The method of clause 32, any other suitable clause, or any combination of suitable clauses, wherein the therapeutic target is JAK (ITK/TXK/JAK3).
56. The method of clause 32, any other suitable clause, or any combination of suitable clauses, wherein the therapeutic target is JAK3/TEC.
57. The method of clause 32, any other suitable clause, or any combination of suitable clauses, wherein the therapeutic target is MC1r.
58. The method of clause 32, any other suitable clause, or any combination of suitable clauses, wherein the therapeutic target is MRP2.
59. The method of clause 32, any other suitable clause, or any combination of suitable clauses, wherein the therapeutic target is NLRP3 inflammasome.
60. The method of clause 32, any other suitable clause, or any combination of suitable clauses, wherein the therapeutic target is NLRX1.
61. The method of clause 32, any other suitable clause, or any combination of suitable clauses, wherein the therapeutic target is PDE4.
62. The method of clause 32, any other suitable clause, or any combination of suitable clauses, wherein the therapeutic target is PSGL-1.
63. The method of clause 32, any other suitable clause, or any combination of suitable clauses, wherein the therapeutic target is RIPK1.
64. The method of clause 32, any other suitable clause, or any combination of suitable clauses, wherein the therapeutic target is S1P1.
65. The method of clause 32, any other suitable clause, or any combination of suitable clauses, wherein the therapeutic target is TLIA.
66. The method of clause 32, any other suitable clause, or any combination of suitable clauses, wherein the therapeutic target is TLR9.
67. The method of clause 32, any other suitable clause, or any combination of suitable clauses, wherein the therapeutic target is TNFa.
68. The method of clause 32, any other suitable clause, or any combination of suitable clauses, wherein the therapeutic target is TNFSF15.
69. The method of clause 32, any other suitable clause, or any combination of suitable clauses, wherein the therapeutic target is TPL2.
70. The method of clause 32, any other suitable clause, or any combination of suitable clauses, wherein the therapeutic target is TREM1.
71. The method of clause 32, any other suitable clause, or any combination of suitable clauses, wherein the therapeutic target is TYK2.
72. The method of clause 1, any other suitable clause, or any combination of suitable clauses, wherein the assessment comprises monitoring of mucosal lesions in the subject.
73. The method of clause 1, any other suitable clause, or any combination of suitable clauses, wherein the assessment comprises evaluation of inflammation in the subject.
74. The method of clause 73, any other suitable clause, or any combination of suitable clauses, wherein the evaluation of inflammation comprises analysis of quantity of inflammation.
75. The method of clause 73, any other suitable clause, or any combination of suitable clauses, wherein the evaluation of inflammation comprises analysis of an increase or a decrease in inflammation.
76. The method of clause 73, any other suitable clause, or any combination of suitable clauses, wherein the evaluation of inflammation comprises analysis of change in inflammation.
77. The method of clause 1, any other suitable clause, or any combination of suitable clauses, wherein the assessment comprises evaluation of a cell type in the subject.
78. The method of clause 77, any other suitable clause, or any combination of suitable clauses, wherein the evaluation comprises analysis of quantity of the cell type.
79. The method of clause 77, any other suitable clause, or any combination of suitable clauses, wherein the evaluation comprises analysis of an increase or a decrease of the cell type.
80. The method of clause 77, any other suitable clause, or any combination of suitable clauses, wherein the evaluation comprises analysis of a change in relative proportion of the cell type.
81. The method of clause 77, any other suitable clause, or any combination of suitable clauses, wherein the cell type is an inflammatory cell.
82. The method of clause 77, any other suitable clause, or any combination of suitable clauses, wherein the cell type is an immunogenic cell.
83. The method of clause 77, any other suitable clause, or any combination of suitable clauses, wherein the cell type is an enterocyte cell.
84. The method of clause 77, any other suitable clause, or any combination of suitable clauses, wherein the cell type is an enterocyte-related cell.
85. The method of clause 77, any other suitable clause, or any combination of suitable clauses, wherein the cell type is a lymphocyte cell.
86. The method of clause 77, any other suitable clause, or any combination of suitable clauses, wherein the cell type is a lymphocyte-related cell.
87. The method of clause 77, any other suitable clause, or any combination of suitable clauses, wherein the cell type is a T cell.
88. The method of clause 77, any other suitable clause, or any combination of suitable clauses, wherein the cell type is an NK cell.
89. The method of clause 77, any other suitable clause, or any combination of suitable clauses, wherein the cell type is a B cell.
90. The method of clause 77, any other suitable clause, or any combination of suitable clauses, wherein the cell type is a macrophage.
91. The method of clause 77, any other suitable clause, or any combination of suitable clauses, wherein the cell type is a neutrophil.
92. The method of clause 77, any other suitable clause, or any combination of suitable clauses, wherein the cell type is an endothelial cell.
93. The method of clause 77, any other suitable clause, or any combination of suitable clauses, wherein the cell type is a fibroblast.
94. The method of clause 1, any other suitable clause, or any combination of suitable clauses, wherein the assessment is a prognosis of therapeutic response to a medical intervention in the subject.
95. The method of clause 94, any other suitable clause, or any combination of suitable clauses, wherein the medical intervention is a drug therapy.
96. The method of clause 95, any other suitable clause, or any combination of suitable clauses, wherein the drug therapy is an IBD drug therapy.
97. The method of clause 95, any other suitable clause, or any combination of suitable clauses, wherein the drug therapy is a UC drug therapy.
98. The method of clause 95, any other suitable clause, or any combination of suitable clauses, wherein the drug therapy is a CD drug therapy.
99. The method of clause 95, any other suitable clause, or any combination of suitable clauses, wherein the drug therapy comprises an anti-inflammatory drug.
100. The method of clause 95, any other suitable clause, or any combination of suitable clauses, wherein the drug therapy comprises an immune system suppressing drug.
101. The method of clause 95, any other suitable clause, or any combination of suitable clauses, wherein the drug therapy comprises a biologic.
102. The method of clause 95, any other suitable clause, or any combination of suitable clauses, wherein the drug therapy comprises an antibiotic.
103. The method of clause 94, any other suitable clause, or any combination of suitable clauses, wherein the medical intervention is a colonoscopy.
104. The method of clause 1, any other suitable clause, or any combination of suitable clauses, wherein the assessment is a determination of therapeutic effectiveness of a medical intervention in the subject.
105. The method of clause 104, any other suitable clause, or any combination of suitable clauses, wherein the medical intervention is a drug therapy.
106. The method of clause 105, any other suitable clause, or any combination of suitable clauses, wherein the drug therapy is an IBD drug therapy.
107. The method of clause 105, any other suitable clause, or any combination of suitable clauses, wherein the drug therapy is a UC drug therapy.
108. The method of clause 105, any other suitable clause, or any combination of suitable clauses, wherein the drug therapy is a CD drug therapy.
109. The method of clause 105, any other suitable clause, or any combination of suitable clauses, wherein the drug therapy comprises an anti-inflammatory drug.
110. The method of clause 105, any other suitable clause, or any combination of suitable clauses, wherein the drug therapy comprises an immune system suppressing drug.
111. The method of clause 105, any other suitable clause, or any combination of suitable clauses, wherein the drug therapy comprises a biologic.
112. The method of clause 105, any other suitable clause, or any combination of suitable clauses, wherein the drug therapy comprises an antibiotic.
113. The method of clause 104, any other suitable clause, or any combination of suitable clauses, wherein the medical intervention is a colonoscopy.
114. The method of clause 1, any other suitable clause, or any combination of suitable clauses, wherein measuring the level of expression of nucleic acid biomarkers comprises nucleic acid extraction from the eukaryotic cells.
115. The method of clause 1, any other suitable clause, or any combination of suitable clauses, wherein measuring the level of expression of nucleic acid biomarkers comprises DNA extraction from the eukaryotic cells.
116. The method of clause 1, any other suitable clause, or any combination of suitable clauses, wherein measuring the level of expression of nucleic acid biomarkers comprises RNA extraction from the eukaryotic cells.
117. The method of clause 1, any other suitable clause, or any combination of suitable clauses, wherein measuring the level of expression of nucleic acid biomarkers comprises next generation sequencing.
118. The method of clause 1, any other suitable clause, or any combination of suitable clauses, wherein the method further comprises diagnosing disease in the subject.
19. The method of clause 118, any other suitable clause, or any combination of suitable clauses, wherein the disease is inflammatory bowel disease (IBD).
120. The method of clause 118, any other suitable clause, or any combination of suitable clauses, wherein the disease is ulcerative colitis (UC).
121. The method of clause 118, any other suitable clause, or any combination of suitable clauses, wherein the disease is Crohn's disease (CD).
122. The method of clause 1, any other suitable clause, or any combination of suitable clauses, wherein the method further comprises treating disease in the subject.
123. The method of clause 122, any other suitable clause, or any combination of suitable clauses, wherein the disease is inflammatory bowel disease (IBD).
124 The method of clause 122, any other suitable clause, or any combination of suitable clauses, wherein the disease is ulcerative colitis (UC).
125. The method of clause 122, any other suitable clause, or any combination of suitable clauses, wherein the disease is Crohn's disease (CD).
126. The method of clause 122, any other suitable clause, or any combination of suitable clauses, wherein treating disease comprises administering an IBD drug therapy to the subject.
127. The method of clause 122, any other suitable clause, or any combination of suitable clauses, wherein treating disease comprises administering a UC drug therapy to the subject.
128. The method of clause 122, any other suitable clause, or any combination of suitable clauses, wherein treating disease comprises administering a CD drug therapy to the subject.
129. The method of clause 122, any other suitable clause, or any combination of suitable clauses, wherein treating disease comprises administering an anti-inflammatory drug to the subject.
130. The method of clause 122, any other suitable clause, or any combination of suitable clauses, wherein treating disease comprises administering an immune system suppressing drug to the subject.
131. The method of clause 122, any other suitable clause, or any combination of suitable clauses, wherein treating disease comprises administering a biologic to the subject.
132. The method of clause 122, any other suitable clause, or any combination of suitable clauses, wherein treating disease comprises administering an antibiotic to the subject.
133. A method of predicting therapeutic response to a disease in a subject, the method comprising:
measuring the level of expression of one or more stool-derived eukaryotic nucleic acid biomarkers selected from the biomarkers described herein in eukaryotic nucleic acid extracted from a stool sample from the subject, wherein the measured expression level of the one or more stool-derived eukaryotic nucleic acid biomarkers in the stool sample indicates therapeutic response to the disease in the subject.
134. The method of clause 133, any other suitable clause, or any combination of suitable clauses, wherein the method comprises comparing the measured expression level of the one or more stool-derived eukaryotic nucleic acid biomarkers in the stool sample with the measured expression level of the one or more stool-derived eukaryotic nucleic acid biomarkers in a control, wherein a difference in the measured expression level of the one or more stool-derived eukaryotic nucleic acid biomarkers in the stool sample relative to the measured expression level of the one or more stool-derived eukaryotic nucleic acid biomarkers in the control indicates therapeutic response to the disease in the subject.
135. The method of clause 133, any other suitable clause, or any combination of suitable clauses, wherein the nucleic acid is DNA.
136. The method of clause 133, any other suitable clause, or any combination of suitable clauses, wherein the nucleic acid is RNA.
137. The method of clause 133, any other suitable clause, or any combination of suitable clauses, wherein the nucleic acid is a combination of DNA and RNA.
138. The method of clause 133, any other suitable clause, or any combination of suitable clauses, wherein the disease is inflammatory bowel disease (IBD).
139. The method of clause 133, any other suitable clause, or any combination of suitable clauses, wherein the disease is ulcerative colitis (UC).
140. The method of clause 133, any other suitable clause, or any combination of suitable clauses, wherein the disease is Crohn's disease (CD).
141. The method of clause 133, any other suitable clause, or any combination of suitable clauses, wherein the therapeutic response comprises an assessment of disease activity in the subject.
142. The method of clause 141, any other suitable clause, or any combination of suitable clauses, wherein the assessment is a determination of active IBD symptoms in the subject.
143. The method of clause 142, any other suitable clause, or any combination of suitable clauses, wherein the determination of active IBD symptoms indicates mild IBD symptoms.
144. The method of clause 142, any other suitable clause, or any combination of suitable clauses, wherein the determination of active IBD symptoms indicates moderate IBD symptoms.
145. The method of clause 142, any other suitable clause, or any combination of suitable clauses, wherein the determination of active IBD symptoms indicates severe IBD symptoms.
146. The method of clause 133, any other suitable clause, or any combination of suitable clauses, wherein the assessment is a determination of IBD remission in the subject.
147. The method of clause 133, any other suitable clause, or any combination of suitable clauses, wherein the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 1.
148. The method of clause 133, any other suitable clause, or any combination of suitable clauses, wherein the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 2.
149. The method of clause 133, any other suitable clause, or any combination of suitable clauses, wherein the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 3.
150. The method of clause 133, any other suitable clause, or any combination of suitable clauses, wherein the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 4.
151. The method of clause 133, any other suitable clause, or any combination of suitable clauses, wherein the stool-derived eukaryotic nucleic acid biomarkers comprise a combination of biomarkers listed in Table 5.
152. The method of clause 133, any other suitable clause, or any combination of suitable clauses, wherein the one or more stool-derived eukaryotic nucleic acid biomarkers comprise stool-derived eukaryotic RNA biomarkers.
153. The method of clause 152, any other suitable clause, or any combination of suitable clauses, wherein the stool-derived eukaryotic RNA biomarkers comprise enterocyte-specific biomarkers.
154. The method of clause 152, any other suitable clause, or any combination of suitable clauses, wherein the stool-derived eukaryotic RNA biomarkers consist essentially of enterocyte-specific biomarkers.
155. The method of clause 152, any other suitable clause, or any combination of suitable clauses, wherein the stool-derived eukaryotic RNA biomarkers consist of enterocyte-specific biomarkers.
156. The method of clause 152, any other suitable clause, or any combination of suitable clauses, wherein the stool-derived eukaryotic RNA biomarkers comprise lymphocyte-specific biomarkers.
157. The method of clause 152, any other suitable clause, or any combination of suitable clauses, wherein the stool-derived eukaryotic RNA biomarkers consist essentially of lymphocyte-specific biomarkers.
158. The method of clause 152, any other suitable clause, or any combination of suitable clauses, wherein the stool-derived eukaryotic RNA biomarkers consist of lymphocyte-specific biomarkers.
159. The method of clause 152, any other suitable clause, or any combination of suitable clauses, wherein the stool-derived eukaryotic RNA biomarkers comprise enterocyte-specific biomarkers and lymphocyte-specific biomarkers.
160. The method of clause 152, any other suitable clause, or any combination of suitable clauses, wherein the stool-derived eukaryotic RNA biomarkers consist essentially of enterocyte-specific biomarkers and lymphocyte-specific biomarkers.
161. The method of clause 152, any other suitable clause, or any combination of suitable clauses, wherein the stool-derived eukaryotic RNA biomarkers consist of enterocyte-specific biomarkers and lymphocyte-specific biomarkers.
162. The method of clause 152, any other suitable clause, or any combination of suitable clauses, wherein the stool-derived eukaryotic RNA biomarkers comprise one or more non-therapeutic targets.
163. The method of clause 162, any other suitable clause, or any combination of suitable clauses, wherein the one or more non-therapeutic targets are selected from the group consisting of RNA transcripts that mediate cellular cytoskeleton remodeling, growth, and inflammatory response.
164. The method of clause 152, any other suitable clause, or any combination of suitable clauses, wherein the stool-derived eukaryotic RNA biomarkers comprise one or more therapeutic targets.
165. The method of clause 164, any other suitable clause, or any combination of suitable clauses, wherein the one or more therapeutic targets are selected from the group consisting of TNF, MADCAMI, ITGA4, JAK1, TYK2, IL-12, IL-23, and any combination thereof.
166. The method of clause 164, any other suitable clause, or any combination of suitable clauses, wherein the therapeutic target is TNF.
167. The method of clause 164, any other suitable clause, or any combination of suitable clauses, wherein the therapeutic target is MADCAM1.
168. The method of clause 164, any other suitable clause, or any combination of suitable clauses, wherein the therapeutic target is ITGA4.
169. The method of clause 164, any other suitable clause, or any combination of suitable clauses, wherein the therapeutic target is JAK1.
170. The method of clause 164, any other suitable clause, or any combination of suitable clauses, wherein the therapeutic target is TYK2.
171. The method of clause 164, any other suitable clause, or any combination of suitable clauses, wherein the therapeutic target is IL-12.
172. The method of clause 164, any other suitable clause, or any combination of suitable clauses, wherein the therapeutic target is IL-23.
173. The method of clause 164, any other suitable clause, or any combination of suitable clauses, wherein the one or more therapeutic targets are selected from the group consisting of a4B7, CCR6, CD30 ligand, FPR1, GLP-2, IL-2, IL-4/IL-13, IL7R, IL-10, IL-12/IL-23, IL-36, JAK, JAKI, JAK (ITK/TXK/JAK3), JAK3/TEC, MC1r, MRP2, NLRP3 inflammasome, NLRX1, PDE4, PSGL-1, RIPK1, S1P1, TLIA, TLR9, TNFa, TNFSF15, TPL2, TREM1, TYK2, and any combination thereof.
174. The method of clause 164, any other suitable clause, or any combination of suitable clauses, wherein the therapeutic target is a4B7.
175. The method of clause 164, any other suitable clause, or any combination of suitable clauses, wherein the therapeutic target is CCR6.
176. The method of clause 164, any other suitable clause, or any combination of suitable clauses, wherein the therapeutic target is CD30 ligand.
177. The method of clause 164, any other suitable clause, or any combination of suitable clauses, wherein the therapeutic target is FPR1.
178. The method of clause 164, any other suitable clause, or any combination of suitable clauses, wherein the therapeutic target is GLP-2.
179. The method of clause 164, any other suitable clause, or any combination of suitable clauses, wherein the therapeutic target is IL-2.
180. The method of clause 164, any other suitable clause, or any combination of suitable clauses, wherein the therapeutic target is IL-4/IL-13.
181. The method of clause 164, any other suitable clause, or any combination of suitable clauses, wherein the therapeutic target is IL7R.
182. The method of clause 164, any other suitable clause, or any combination of suitable clauses, wherein the therapeutic target is IL-10.
183. The method of clause 164, any other suitable clause, or any combination of suitable clauses, wherein the therapeutic target is IL-12/IL-23.
184. The method of clause 164, any other suitable clause, or any combination of suitable clauses, wherein the therapeutic target is IL-36.
185. The method of clause 164, any other suitable clause, or any combination of suitable clauses, wherein the therapeutic target is JAK.
186. The method of clause 164, any other suitable clause, or any combination of suitable clauses, wherein the therapeutic target is JAK1.
187. The method of clause 164, any other suitable clause, or any combination of suitable clauses, wherein the therapeutic target is JAK (ITK/TXK/JAK3).
188. The method of clause 164, any other suitable clause, or any combination of suitable clauses, wherein the therapeutic target is JAK3/TEC.
189. The method of clause 164, any other suitable clause, or any combination of suitable clauses, wherein the therapeutic target is MC1r.
90. The method of clause 164, any other suitable clause, or any combination of suitable clauses, wherein the therapeutic target is MRP2.
191. The method of clause 164, any other suitable clause, or any combination of suitable clauses, wherein the therapeutic target is NLRP3 inflammasome.
192. The method of clause 164, any other suitable clause, or any combination of suitable clauses, wherein the therapeutic target is NLRX1.
193. The method of clause 164, any other suitable clause, or any combination of suitable clauses, wherein the therapeutic target is PDE4.
194. The method of clause 164, any other suitable clause, or any combination of suitable clauses, wherein the therapeutic target is PSGL-1.
195. The method of clause 164, any other suitable clause, or any combination of suitable clauses, wherein the therapeutic target is RIPK1.
196. The method of clause 164, any other suitable clause, or any combination of suitable clauses, wherein the therapeutic target is S1P1.
197. The method of clause 164, any other suitable clause, or any combination of suitable clauses, wherein the therapeutic target is TL1A.
198. The method of clause 164, any other suitable clause, or any combination of suitable clauses, wherein the therapeutic target is TLR9.
199. The method of clause 164, any other suitable clause, or any combination of suitable clauses, wherein the therapeutic target is TNFa.
200 The method of clause 164, any other suitable clause, or any combination of suitable clauses, wherein the therapeutic target is TNFSF15.
201. The method of clause 164, any other suitable clause, or any combination of suitable clauses, wherein the therapeutic target is TPL2.
202. The method of clause 164, any other suitable clause, or any combination of suitable clauses, wherein the therapeutic target is TREM1.
203. The method of clause 164, any other suitable clause, or any combination of suitable clauses, wherein the therapeutic target is TYK2.
204. The method of clause 133, any other suitable clause, or any combination of suitable clauses, wherein the assessment comprises evaluation of inflammation in the subject.
205. The method of clause 204, any other suitable clause, or any combination of suitable clauses, wherein the evaluation of inflammation comprises analysis of quantity of inflammation.
206. The method of clause 204, any other suitable clause, or any combination of suitable clauses, wherein the evaluation of inflammation comprises analysis of an increase or a decrease in inflammation.
207. The method of clause 204, any other suitable clause, or any combination of suitable clauses, wherein the evaluation of inflammation comprises analysis of change in inflammation.
208. The method of clause 133, any other suitable clause, or any combination of suitable clauses, wherein the assessment comprises evaluation of a cell type in the subject. 209. The method of clause 208, any other suitable clause, or any combination of suitable clauses, wherein the evaluation comprises analysis of quantity of the cell type.
210. The method of clause 208, any other suitable clause, or any combination of suitable clauses, wherein the evaluation comprises analysis of an increase or a decrease of the cell type.
211. The method of clause 208, any other suitable clause, or any combination of suitable clauses, wherein the evaluation comprises analysis of a change in relative proportion of the cell type.
212. The method of clause 208, any other suitable clause, or any combination of suitable clauses, wherein the cell type is an inflammatory cell.
213. The method of clause 208, any other suitable clause, or any combination of suitable clauses, wherein the cell type is an immunogenic cell.
214. The method of clause 208, any other suitable clause, or any combination of suitable clauses, wherein the cell type is an enterocyte cell.
215. The method of clause 208, any other suitable clause, or any combination of suitable clauses, wherein the cell type is an enterocyte-related cell.
216. The method of clause 208, any other suitable clause, or any combination of suitable clauses, wherein the cell type is a lymphocyte cell.
217. The method of clause 208, any other suitable clause, or any combination of suitable clauses, wherein the cell type is a lymphocyte-related cell.
218. The method of clause 208, any other suitable clause, or any combination of suitable clauses, wherein the cell type is a T cell.
219. The method of clause 208, any other suitable clause, or any combination of suitable clauses, wherein the cell type is an NK cell.
220. The method of clause 208, any other suitable clause, or any combination of suitable clauses, wherein the cell type is a B cell.
221. The method of clause 208, any other suitable clause, or any combination of suitable clauses, wherein the cell type is a macrophage.
222. The method of clause 208, any other suitable clause, or any combination of suitable clauses, wherein the cell type is a neutrophil.
223. The method of clause 208, any other suitable clause, or any combination of suitable clauses, wherein the cell type is an endothelial cell.
224. The method of clause 208, any other suitable clause, or any combination of suitable clauses, wherein the cell type is a fibroblast.
225. The method of clause 133, any other suitable clause, or any combination of suitable clauses, wherein the therapeutic response is to a medical intervention in the subject.
226. The method of clause 225, any other suitable clause, or any combination of suitable clauses, wherein the medical intervention is a drug therapy.
227. The method of clause 226, any other suitable clause, or any combination of suitable clauses, wherein the drug therapy is an IBD drug therapy.
228. The method of clause 226, any other suitable clause, or any combination of suitable clauses, wherein the drug therapy is a UC drug therapy.
229. The method of clause 226, any other suitable clause, or any combination of suitable clauses, wherein the drug therapy is a CD drug therapy.
230. The method of clause 226, any other suitable clause, or any combination of suitable clauses, wherein the drug therapy comprises an anti-inflammatory drug.
231. The method of clause 226, any other suitable clause, or any combination of suitable clauses, wherein the drug therapy comprises an immune system suppressing drug.
232. The method of clause 226, any other suitable clause, or any combination of suitable clauses, wherein the drug therapy comprises a biologic.
233. The method of clause 226, any other suitable clause, or any combination of suitable clauses, wherein the drug therapy comprises an antibiotic.
234. The method of clause 225, any other suitable clause, or any combination of suitable clauses, wherein the medical intervention is a colonoscopy.
235. The method of clause 133, any other suitable clause, or any combination of suitable clauses, wherein measuring the level of expression of nucleic acid biomarkers comprises nucleic acid extraction from the eukaryotic cells.
236. The method of clause 133, any other suitable clause, or any combination of suitable clauses, wherein measuring the level of expression of nucleic acid biomarkers comprises DNA extraction from the eukaryotic cells.
237. The method of clause 133, any other suitable clause, or any combination of suitable clauses, wherein measuring the level of expression of nucleic acid biomarkers comprises RNA extraction from the eukaryotic cells.
238. The method of clause 133, any other suitable clause, or any combination of suitable clauses, wherein measuring the level of expression of nucleic acid biomarkers comprises next generation sequencing.
239. The method of clause 133, any other suitable clause, or any combination of suitable clauses, wherein the method further comprises c) treating disease in the subject.
240. The method of clause 239, any other suitable clause, or any combination of suitable clauses, wherein the disease is inflammatory bowel disease (IBD).
241. The method of clause 239, any other suitable clause, or any combination of suitable clauses, wherein the disease is ulcerative colitis (UC).
242. The method of clause 239, any other suitable clause, or any combination of suitable clauses, wherein the disease is Crohn's disease (CD).
243. The method of clause 239, any other suitable clause, or any combination of suitable clauses, wherein treating disease comprises administering an IBD drug therapy to the subject.
244. The method of clause 239, any other suitable clause, or any combination of suitable clauses, wherein treating disease comprises administering a UC drug therapy to the subject.
245. The method of clause 239, any other suitable clause, or any combination of suitable clauses, wherein treating disease comprises administering a CD drug therapy to the subject.
246. The method of clause 239, any other suitable clause, or any combination of suitable clauses, wherein treating disease comprises administering an anti-inflammatory drug to the subject.
247. The method of clause 239, any other suitable clause, or any combination of suitable clauses, wherein treating disease comprises administering an immune system suppressing drug to the subject.
248. The method of clause 239, any other suitable clause, or any combination of suitable clauses, wherein treating disease comprises administering a biologic to the subject.
249. The method of clause 239, any other suitable clause, or any combination of suitable clauses, wherein treating disease comprises administering an antibiotic to the subject.
250. A method of assessing inflammation in a subject, the method comprising: measuring the level of expression of one or more stool-derived eukaryotic nucleic acid biomarkers selected from the biomarkers described herein in eukaryotic nucleic acid extracted from a stool sample from the subject, wherein the measured expression level of the one or more stool-derived eukaryotic nucleic acid biomarkers in the stool sample indicates the assessment of inflammation in the subject.
251. The method of clause 250, any other suitable clause, or any combination of suitable clauses, wherein the method comprises comparing the measured expression level of the one or more stool-derived eukaryotic nucleic acid biomarkers in the stool sample with the measured expression level of the one or more stool-derived eukaryotic nucleic acid biomarkers in a control, wherein a difference in the measured expression level of the one or more stool-derived eukaryotic nucleic acid biomarkers in the stool sample relative to the measured expression level of the one or more stool-derived eukaryotic nucleic acid biomarkers in the control indicates the assessment of inflammation in the subject.
252. The method of clause 250, any other suitable clause, or any combination of suitable clauses, wherein the nucleic acid is DNA.
253. The method of clause 250, any other suitable clause, or any combination of suitable clauses, wherein the nucleic acid is RNA.
254. The method of clause 250, any other suitable clause, or any combination of suitable clauses, wherein the nucleic acid is a combination of DNA and RNA.
255. The method of clause 250, any other suitable clause, or any combination of suitable clauses, wherein the inflammation is associated with a disease.
256. The method of clause 255, any other suitable clause, or any combination of suitable clauses, wherein the disease is inflammatory bowel disease (IBD).
257. The method of clause 255, any other suitable clause, or any combination of suitable clauses, wherein the disease is ulcerative colitis (UC).
258. The method of clause 255, any other suitable clause, or any combination of suitable clauses, wherein the disease is Crohn's disease (CD).
259. The method of clause 250, any other suitable clause, or any combination of suitable clauses, wherein the assessment of inflammation comprises an assessment of disease activity in the subject.
260. The method of clause 259, any other suitable clause, or any combination of suitable clauses, wherein the assessment is a determination of active IBD symptoms in the subject.
261. The method of clause 260, any other suitable clause, or any combination of suitable clauses, wherein the determination of active IBD symptoms indicates mild IBD symptoms.
262. The method of clause 260, any other suitable clause, or any combination of suitable clauses, wherein the determination of active IBD symptoms indicates moderate IBD symptoms.
263. The method of clause 260, any other suitable clause, or any combination of suitable clauses, wherein the determination of active IBD symptoms indicates severe IBD symptoms.
264. The method of clause 259, any other suitable clause, or any combination of suitable clauses, wherein the assessment is a determination of IBD remission in the subject.
265. The method of clause 250, any other suitable clause, or any combination of suitable clauses, wherein the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 1.
266. The method of clause 250, any other suitable clause, or any combination of suitable clauses, wherein the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 2.
267. The method of clause 250, any other suitable clause, or any combination of suitable clauses, wherein the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 3.
268. The method of clause 250, any other suitable clause, or any combination of suitable clauses, wherein the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 4.
269. The method of clause 250, any other suitable clause, or any combination of suitable clauses, wherein the stool-derived eukaryotic nucleic acid biomarkers comprise a combination of biomarkers listed in Table 5.
270. The method of clause 250, any other suitable clause, or any combination of suitable clauses, wherein the one or more stool-derived eukaryotic nucleic acid biomarkers comprise stool-derived eukaryotic RNA biomarkers.
271. The method of clause 270, any other suitable clause, or any combination of suitable clauses, wherein the stool-derived eukaryotic RNA biomarkers comprise enterocyte-specific biomarkers.
272. The method of clause 270, any other suitable clause, or any combination of suitable clauses, wherein the stool-derived eukaryotic RNA biomarkers consist essentially of enterocyte-specific biomarkers.
273. The method of clause 270, any other suitable clause, or any combination of suitable clauses, wherein the stool-derived eukaryotic RNA biomarkers consist of enterocyte-specific biomarkers.
274. The method of clause 270, any other suitable clause, or any combination of suitable clauses, wherein the stool-derived eukaryotic RNA biomarkers comprise lymphocyte-specific biomarkers.
275. The method of clause 270, any other suitable clause, or any combination of suitable clauses, wherein the stool-derived eukaryotic RNA biomarkers consist essentially of lymphocyte-specific biomarkers.
276. The method of clause 270, any other suitable clause, or any combination of suitable clauses, wherein the stool-derived eukaryotic RNA biomarkers consist of lymphocyte-specific biomarkers.
277. The method of clause 270, any other suitable clause, or any combination of suitable clauses, wherein the stool-derived eukaryotic RNA biomarkers comprise enterocyte-specific biomarkers and lymphocyte-specific biomarkers.
278. The method of clause 270, any other suitable clause, or any combination of suitable clauses, wherein the stool-derived eukaryotic RNA biomarkers consist essentially of enterocyte-specific biomarkers and lymphocyte-specific biomarkers.
279. The method of clause 270, any other suitable clause, or any combination of suitable clauses, wherein the stool-derived eukaryotic RNA biomarkers consist of enterocyte-specific biomarkers and lymphocyte-specific biomarkers.
280. The method of clause 270, any other suitable clause, or any combination of suitable clauses, wherein the stool-derived eukaryotic RNA biomarkers comprise one or more non-therapeutic targets.
281. The method of clause 280, any other suitable clause, or any combination of suitable clauses, wherein the one or more non-therapeutic targets are selected from the group consisting of RNA transcripts that mediate cellular cytoskeleton remodeling, growth, and inflammatory response.
282. The method of clause 270, any other suitable clause, or any combination of suitable clauses, wherein the stool-derived eukaryotic RNA biomarkers comprise one or more therapeutic targets.
283. The method of clause 282, any other suitable clause, or any combination of suitable clauses, wherein the one or more therapeutic targets are selected from the group consisting of TNF, MADCAMI, ITGA4, JAKI, TYK2, IL-12, IL-23, and any combination thereof.
284. The method of clause 282, any other suitable clause, or any combination of suitable clauses, wherein the therapeutic target is TNF.
285. The method of clause 282, any other suitable clause, or any combination of suitable clauses, wherein the therapeutic target is MADCAM1.
286. The method of clause 282, any other suitable clause, or any combination of suitable clauses, wherein the therapeutic target is ITGA4.
287. The method of clause 282, any other suitable clause, or any combination of suitable clauses, wherein the therapeutic target is JAK1.
288. The method of clause 282, any other suitable clause, or any combination of suitable clauses, wherein the therapeutic target is TYK2.
289. The method of clause 282, any other suitable clause, or any combination of suitable clauses, wherein the therapeutic target is IL-12.
290. The method of clause 282, any other suitable clause, or any combination of suitable clauses, wherein the therapeutic target is IL-23.
291. The method of clause 282, any other suitable clause, or any combination of suitable clauses, wherein the one or more therapeutic targets are selected from the group consisting of a4B7, CCR6, CD30 ligand, FPR1, GLP-2, IL-2, IL-4/IL-13, IL7R, IL-10, IL-12/IL-23, IL-36, JAK, JAKI, JAK (ITK/TXK/JAK3), JAK3/TEC, MC1r, MRP2, NLRP3 inflammasome, NLRX1, PDE4, PSGL-1, RIPK1, S1P1, TLIA, TLR9, TNFa, TNFSF15, TPL2, TREM1, TYK2, and any combination thereof.
292. The method of clause 282, any other suitable clause, or any combination of suitable clauses, wherein the therapeutic target is a4B7.
293. The method of clause 282, any other suitable clause, or any combination of suitable clauses, wherein the therapeutic target is CCR6.
294. The method of clause 282, any other suitable clause, or any combination of suitable clauses, wherein the therapeutic target is CD30 ligand.
295. The method of clause 282, any other suitable clause, or any combination of suitable clauses, wherein the therapeutic target is FPR1.
296. The method of clause 282, any other suitable clause, or any combination of suitable clauses, wherein the therapeutic target is GLP-2.
297. The method of clause 282, any other suitable clause, or any combination of suitable clauses, wherein the therapeutic target is IL-2.
298. The method of clause 282, any other suitable clause, or any combination of suitable clauses, wherein the therapeutic target is IL-4/IL-13.
299. The method of clause 282, any other suitable clause, or any combination of suitable clauses, wherein the therapeutic target is IL7R.
300 The method of clause 282, any other suitable clause, or any combination of suitable clauses, wherein the therapeutic target is IL-10.
301. The method of clause 282, any other suitable clause, or any combination of suitable clauses, wherein the therapeutic target is IL-12/IL-23.
302. The method of clause 282, any other suitable clause, or any combination of suitable clauses, wherein the therapeutic target is IL-36.
303. The method of clause 282, any other suitable clause, or any combination of suitable clauses, wherein the therapeutic target is JAK.
304. The method of clause 282, any other suitable clause, or any combination of suitable clauses, wherein the therapeutic target is JAK1.
305. The method of clause 282, any other suitable clause, or any combination of suitable clauses, wherein the therapeutic target is JAK (ITK/TXK/JAK3).
306. The method of clause 282, any other suitable clause, or any combination of suitable clauses, wherein the therapeutic target is JAK3/TEC.
307. The method of clause 282, any other suitable clause, or any combination of suitable clauses, wherein the therapeutic target is MC1r.
308. The method of clause 282, any other suitable clause, or any combination of suitable clauses, wherein the therapeutic target is MRP2.
309. The method of clause 282, any other suitable clause, or any combination of suitable clauses, wherein the therapeutic target is NLRP3 inflammasome.
310. The method of clause 282, any other suitable clause, or any combination of suitable clauses, wherein the therapeutic target is NLRX1.
311. The method of clause 282, any other suitable clause, or any combination of suitable clauses, wherein the therapeutic target is PDE4.
312. The method of clause 282, any other suitable clause, or any combination of suitable clauses, wherein the therapeutic target is PSGL-1.
313. The method of clause 282, any other suitable clause, or any combination of suitable clauses, wherein the therapeutic target is RIPK1.
314. The method of clause 282, any other suitable clause, or any combination of suitable clauses, wherein the therapeutic target is S1P1.
315. The method of clause 282, any other suitable clause, or any combination of suitable clauses, wherein the therapeutic target is TLIA.
316. The method of clause 282, any other suitable clause, or any combination of suitable clauses, wherein the therapeutic target is TLR9.
317. The method of clause 282, any other suitable clause, or any combination of suitable clauses, wherein the therapeutic target is TNFa.
318. The method of clause 282, any other suitable clause, or any combination of suitable clauses, wherein the therapeutic target is TNFSF15.
319. The method of clause 282, any other suitable clause, or any combination of suitable clauses, wherein the therapeutic target is TPL2.
320. The method of clause 282, any other suitable clause, or any combination of suitable clauses, wherein the therapeutic target is TREM1.
321. The method of clause 282, any other suitable clause, or any combination of suitable clauses, wherein the therapeutic target is TYK2.
322. The method of clause 250, any other suitable clause, or any combination of suitable clauses, wherein the assessment comprises monitoring of mucosal lesions in the subject.
323. The method of clause 250, any other suitable clause, or any combination of suitable clauses, wherein the assessment comprises evaluation of inflammation in the subject.
324. The method of clause 323, any other suitable clause, or any combination of suitable clauses, wherein the evaluation of inflammation comprises analysis of quantity of inflammation.
325. The method of clause 323, any other suitable clause, or any combination of suitable clauses, wherein the evaluation of inflammation comprises analysis of an increase or a decrease in inflammation.
326. The method of clause 323, any other suitable clause, or any combination of suitable clauses, wherein the evaluation of inflammation comprises analysis of change in inflammation.
327. The method of clause 250, any other suitable clause, or any combination of suitable clauses, wherein the assessment comprises evaluation of a cell type in the subject. 328. The method of clause 327, any other suitable clause, or any combination of suitable clauses, wherein the evaluation comprises analysis of quantity of the cell type.
329. The method of clause 327, any other suitable clause, or any combination of suitable clauses, wherein the evaluation comprises analysis of an increase or a decrease of the cell type.
330. The method of clause 327, any other suitable clause, or any combination of suitable clauses, wherein the evaluation comprises analysis of a change in relative proportion of the cell type.
331. The method of clause 327, any other suitable clause, or any combination of suitable clauses, wherein the cell type is an inflammatory cell.
332. The method of clause 327, any other suitable clause, or any combination of suitable clauses, wherein the cell type is an immunogenic cell.
333. The method of clause 327, any other suitable clause, or any combination of suitable clauses, wherein the cell type is an enterocyte cell.
334. The method of clause 327, any other suitable clause, or any combination of suitable clauses, wherein the cell type is an enterocyte-related cell.
335. The method of clause 327, any other suitable clause, or any combination of suitable clauses, wherein the cell type is a lymphocyte cell.
336. The method of clause 327, any other suitable clause, or any combination of suitable clauses, wherein the cell type is a lymphocyte-related cell.
337. The method of clause 327, any other suitable clause, or any combination of suitable clauses, wherein the cell type is a T cell.
338. The method of clause 327, any other suitable clause, or any combination of suitable clauses, wherein the cell type is an NK cell.
339. The method of clause 327, any other suitable clause, or any combination of suitable clauses, wherein the cell type is a B cell.
340. The method of clause 327, any other suitable clause, or any combination of suitable clauses, wherein the cell type is a macrophage.
341. The method of clause 327, any other suitable clause, or any combination of suitable clauses, wherein the cell type is a neutrophil.
342. The method of clause 327, any other suitable clause, or any combination of suitable clauses, wherein the cell type is an endothelial cell.
343. The method of clause 327, any other suitable clause, or any combination of suitable clauses, wherein the cell type is a fibroblast.
344. The method of clause 250, any other suitable clause, or any combination of suitable clauses, wherein the assessment comprises a prognosis of therapeutic response to a medical intervention in the subject.
345. The method of clause 344, any other suitable clause, or any combination of suitable clauses, wherein the medical intervention is a drug therapy.
346. The method of clause 345, any other suitable clause, or any combination of suitable clauses, wherein the drug therapy is an IBD drug therapy.
347. The method of clause 345, any other suitable clause, or any combination of suitable clauses, wherein the drug therapy is a UC drug therapy.
348. The method of clause 345, any other suitable clause, or any combination of suitable clauses, wherein the drug therapy is a CD drug therapy.
349. The method of clause 345, any other suitable clause, or any combination of suitable clauses, wherein the drug therapy comprises an anti-inflammatory drug.
350. The method of clause 345, any other suitable clause, or any combination of suitable clauses, wherein the drug therapy comprises an immune system suppressing drug.
351. The method of clause 345, any other suitable clause, or any combination of suitable clauses, wherein the drug therapy comprises a biologic.
352. The method of clause 345, any other suitable clause, or any combination of suitable clauses, wherein the drug therapy comprises an antibiotic.
353. The method of clause 344, any other suitable clause, or any combination of suitable clauses, wherein the medical intervention is a colonoscopy.
354. The method of clause 250, any other suitable clause, or any combination of suitable clauses, wherein the assessment comprises a determination of therapeutic effectiveness of a medical intervention in the subject.
355. The method of clause 354, any other suitable clause, or any combination of suitable clauses, wherein the medical intervention is a drug therapy.
356. The method of clause 355, any other suitable clause, or any combination of suitable clauses, wherein the drug therapy is an IBD drug therapy.
357. The method of clause 355, any other suitable clause, or any combination of suitable clauses, wherein the drug therapy is a UC drug therapy.
358. The method of clause 355, any other suitable clause, or any combination of suitable clauses, wherein the drug therapy is a CD drug therapy.
359. The method of clause 355, any other suitable clause, or any combination of suitable clauses, wherein the drug therapy comprises an anti-inflammatory drug.
360. The method of clause 355, any other suitable clause, or any combination of suitable clauses, wherein the drug therapy comprises an immune system suppressing drug.
361. The method of clause 355, any other suitable clause, or any combination of suitable clauses, wherein the drug therapy comprises a biologic.
362. The method of clause 355, any other suitable clause, or any combination of suitable clauses, wherein the drug therapy comprises an antibiotic.
363. The method of clause 354, any other suitable clause, or any combination of suitable clauses, wherein the medical intervention is a colonoscopy.
364. The method of clause 250, any other suitable clause, or any combination of suitable clauses, wherein measuring the level of expression of nucleic acid biomarkers comprises nucleic acid extraction from the eukaryotic cells.
365. The method of clause 250, any other suitable clause, or any combination of suitable clauses, wherein measuring the level of expression of nucleic acid biomarkers comprises DNA extraction from the eukaryotic cells.
366. The method of clause 250, any other suitable clause, or any combination of suitable clauses, wherein measuring the level of expression of nucleic acid biomarkers comprises RNA extraction from the eukaryotic cells.
367. The method of clause 250, any other suitable clause, or any combination of suitable clauses, wherein measuring the level of expression of nucleic acid biomarkers comprises next generation sequencing.
368. The method of clause 250, any other suitable clause, or any combination of suitable clauses, wherein the method further comprises c) diagnosing disease in the subject.
369. The method of clause 368, any other suitable clause, or any combination of suitable clauses, wherein the disease is inflammatory bowel disease (IBD).
370. The method of clause 368, any other suitable clause, or any combination of suitable clauses, wherein the disease is ulcerative colitis (UC).
371. The method of clause 368, any other suitable clause, or any combination of suitable clauses, wherein the disease is Crohn's disease (CD).
372. The method of clause 250, any other suitable clause, or any combination of suitable clauses, wherein the method further comprises c) treating disease in the subject.
373. The method of clause 372, any other suitable clause, or any combination of suitable clauses, wherein the disease is inflammatory bowel disease (IBD).
374. The method of clause 372, any other suitable clause, or any combination of suitable clauses, wherein the disease is ulcerative colitis (UC).
375. The method of clause 372, any other suitable clause, or any combination of suitable clauses, wherein the disease is Crohn's disease (CD).
376. The method of clause 372, any other suitable clause, or any combination of suitable clauses, wherein treating disease comprises administering an IBD drug therapy to the subject.
377. The method of clause 372, any other suitable clause, or any combination of suitable clauses, wherein treating disease comprises administering a UC drug therapy to the subject.
378. The method of clause 372, any other suitable clause, or any combination of suitable clauses, wherein treating disease comprises administering a CD drug therapy to the subject.
379. The method of clause 372, any other suitable clause, or any combination of suitable clauses, wherein treating disease comprises administering an anti-inflammatory drug to the subject.
380. The method of clause 372, any other suitable clause, or any combination of suitable clauses, wherein treating disease comprises administering an immune system suppressing drug to the subject.
381. The method of clause 372, any other suitable clause, or any combination of suitable clauses, wherein treating disease comprises administering a biologic to the subject.
382. The method of clause 372, any other suitable clause, or any combination of suitable clauses, wherein treating disease comprises administering an antibiotic to the subject.
383. A method of assessing one or more immune cells in a subject, the method comprising:
   measuring the level of expression of one or more stool-derived eukaryotic nucleic acid biomarkers selected from the biomarkers described herein in eukaryotic nucleic acid extracted from a stool sample from the subject;
   wherein the measured expression level of the one or more stool-derived eukaryotic nucleic acid biomarkers in the stool sample indicates the assessment of the one or more immune cells in the subject.
384. The method of clause 383, any other suitable clause, or any combination of suitable clauses, wherein the method comprises comparing the measured expression level of the one or more stool-derived eukaryotic nucleic acid biomarkers in the stool sample with the measured expression level of the one or more stool-derived eukaryotic nucleic acid biomarkers in a control, wherein a difference in the measured expression level of the one or more stool-derived eukaryotic nucleic acid biomarkers in the stool sample relative to the measured expression level of the one or more stool-derived eukaryotic nucleic acid biomarkers in the control indicates the assessment of the immune cell in the subject.
385. The method of clause 383, any other suitable clause, or any combination of suitable clauses, wherein the nucleic acid is DNA.
386. The method of clause 383, any other suitable clause, or any combination of suitable clauses, wherein the nucleic acid is RNA.
387. The method of clause 383, any other suitable clause, or any combination of suitable clauses, wherein the nucleic acid is a combination of DNA and RNA.
388. The method of clause 383, any other suitable clause, or any combination of suitable clauses, wherein the immune cell is associated with a disease.
389. The method of clause 388, any other suitable clause, or any combination of suitable clauses, wherein the disease is inflammatory bowel disease (IBD).
390. The method of clause 388, any other suitable clause, or any combination of suitable clauses, wherein the disease is ulcerative colitis (UC).
391. The method of clause 388, any other suitable clause, or any combination of suitable clauses, wherein the disease is Crohn's disease (CD).
392. The method of clause 383, any other suitable clause, or any combination of suitable clauses, wherein the assessment of immune cells comprises an assessment of disease activity in the subject.
393. The method of clause 392, any other suitable clause, or any combination of suitable clauses, wherein the assessment is a determination of active IBD symptoms in the subject.
394. The method of clause 393, any other suitable clause, or any combination of suitable clauses, wherein the determination of active IBD symptoms indicates mild IBD symptoms.
395. The method of clause 393, any other suitable clause, or any combination of suitable clauses, wherein the determination of active IBD symptoms indicates moderate IBD symptoms.
396. The method of clause 393, any other suitable clause, or any combination of suitable clauses, wherein the determination of active IBD symptoms indicates severe IBD symptoms.
397. The method of clause 392, any other suitable clause, or any combination of suitable clauses, wherein the assessment is a determination of IBD remission in the subject.
398. The method of clause 383, any other suitable clause, or any combination of suitable clauses, wherein the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 1.
399. The method of clause 383, any other suitable clause, or any combination of suitable clauses, wherein the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 2.
400. The method of clause 383, any other suitable clause, or any combination of suitable clauses, wherein the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 3.
401. The method of clause 383, any other suitable clause, or any combination of suitable clauses, wherein the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 4.
402. The method of clause 383, any other suitable clause, or any combination of suitable clauses, wherein the stool-derived eukaryotic nucleic acid biomarkers comprise a combination of biomarkers listed in Table 5.
403. The method of clause 383, any other suitable clause, or any combination of suitable clauses, wherein the one or more stool-derived eukaryotic nucleic acid biomarkers comprise stool-derived eukaryotic RNA biomarkers.
404. The method of clause 403, any other suitable clause, or any combination of suitable clauses, wherein the stool-derived eukaryotic RNA biomarkers comprise enterocyte-specific biomarkers.
405. The method of clause 403, any other suitable clause, or any combination of suitable clauses, wherein the stool-derived eukaryotic RNA biomarkers consist essentially of enterocyte-specific biomarkers.
406. The method of clause 403, any other suitable clause, or any combination of suitable clauses, wherein the stool-derived eukaryotic RNA biomarkers consist of enterocyte-specific biomarkers.
407. The method of clause 403, any other suitable clause, or any combination of suitable clauses, wherein the stool-derived eukaryotic RNA biomarkers comprise lymphocyte-specific biomarkers.
408. The method of clause 403, any other suitable clause, or any combination of suitable clauses, wherein the stool-derived eukaryotic RNA biomarkers consist essentially of lymphocyte-specific biomarkers.
409. The method of clause 403, any other suitable clause, or any combination of suitable clauses, wherein the stool-derived eukaryotic RNA biomarkers consist of lymphocyte-specific biomarkers.
410. The method of clause 403, any other suitable clause, or any combination of suitable clauses, wherein the stool-derived eukaryotic RNA biomarkers comprise enterocyte-specific biomarkers and lymphocyte-specific biomarkers.
411. The method of clause 403, any other suitable clause, or any combination of suitable clauses, wherein the stool-derived eukaryotic RNA biomarkers consist essentially of enterocyte-specific biomarkers and lymphocyte-specific biomarkers.
412. The method of clause 403, any other suitable clause, or any combination of suitable clauses, wherein the stool-derived eukaryotic RNA biomarkers consist of enterocyte-specific biomarkers and lymphocyte-specific biomarkers.
413. The method of clause 403, any other suitable clause, or any combination of suitable clauses, wherein the stool-derived eukaryotic RNA biomarkers comprise one or more non-therapeutic targets.
414. The method of clause 413, any other suitable clause, or any combination of suitable clauses, wherein the one or more non-therapeutic targets are selected from the group consisting of RNA transcripts that mediate cellular cytoskeleton remodeling, growth, and inflammatory response.
415. The method of clause 403, any other suitable clause, or any combination of suitable clauses, wherein the stool-derived eukaryotic RNA biomarkers comprise one or more therapeutic targets.
416. The method of clause 415, any other suitable clause, or any combination of suitable clauses, wherein the one or more therapeutic targets are selected from the group consisting of TNF, MADCAMI, ITGA4, JAK1, TYK2, IL-12, IL-23, and any combination thereof.
417. The method of clause 415, any other suitable clause, or any combination of suitable clauses, wherein the therapeutic target is TNF.
418. The method of clause 415, any other suitable clause, or any combination of suitable clauses, wherein the therapeutic target is MADCAM1.
419. The method of clause 415, any other suitable clause, or any combination of suitable clauses, wherein the therapeutic target is ITGA4.
420. The method of clause 415, any other suitable clause, or any combination of suitable clauses, wherein the therapeutic target is JAK1.
421. The method of clause 415, any other suitable clause, or any combination of suitable clauses, wherein the therapeutic target is TYK2.
422. The method of clause 415, any other suitable clause, or any combination of suitable clauses, wherein the therapeutic target is IL-12.
423. The method of clause 415, any other suitable clause, or any combination of suitable clauses, wherein the therapeutic target is IL-23.
424. The method of clause 415, any other suitable clause, or any combination of suitable clauses, wherein the one or more therapeutic targets are selected from the group consisting of a4B7, CCR6, CD30 ligand, FPR1, GLP-2, IL-2, IL-4/IL-13, IL7R, IL-10, IL-12/IL-23, IL-36, JAK, JAKI, JAK (ITK/TXK/JAK3), JAK3/TEC, MC1r, MRP2, NLRP3 inflammasome, NLRX1, PDE4, PSGL-1, RIPK1, S1P1, TLIA, TLR9, TNFa, TNFSF15, TPL2, TREM1, TYK2, and any combination thereof.
425. The method of clause 415, any other suitable clause, or any combination of suitable clauses, wherein the therapeutic target is a4B7.
426. The method of clause 415, any other suitable clause, or any combination of suitable clauses, wherein the therapeutic target is CCR6.
427. The method of clause 415, any other suitable clause, or any combination of suitable clauses, wherein the therapeutic target is CD30 ligand.
428. The method of clause 415, any other suitable clause, or any combination of suitable clauses, wherein the therapeutic target is FPR1.
429. The method of clause 415, any other suitable clause, or any combination of suitable clauses, wherein the therapeutic target is GLP-2.
430. The method of clause 415, any other suitable clause, or any combination of suitable clauses, wherein the therapeutic target is IL-2.
431. The method of clause 415, any other suitable clause, or any combination of suitable clauses, wherein the therapeutic target is IL-4/IL-13.
432. The method of clause 415, any other suitable clause, or any combination of suitable clauses, wherein the therapeutic target is IL7R.
433. The method of clause 415, any other suitable clause, or any combination of suitable clauses, wherein the therapeutic target is IL-10.
434. The method of clause 415, any other suitable clause, or any combination of suitable clauses, wherein the therapeutic target is IL-12/IL-23.
435. The method of clause 415, any other suitable clause, or any combination of suitable clauses, wherein the therapeutic target is IL-36.
436. The method of clause 415, any other suitable clause, or any combination of suitable clauses, wherein the therapeutic target is JAK.
437. The method of clause 415, any other suitable clause, or any combination of suitable clauses, wherein the therapeutic target is JAK1.
438. The method of clause 415, any other suitable clause, or any combination of suitable clauses, wherein the therapeutic target is JAK (ITK/TXK/JAK3).
439. The method of clause 415, any other suitable clause, or any combination of suitable clauses, wherein the therapeutic target is JAK3/TEC.
440. The method of clause 415, any other suitable clause, or any combination of suitable clauses, wherein the therapeutic target is MC1r.
441. The method of clause 415, any other suitable clause, or any combination of suitable clauses, wherein the therapeutic target is MRP2.
442. The method of clause 415, any other suitable clause, or any combination of suitable clauses, wherein the therapeutic target is NLRP3 inflammasome.
443. The method of clause 415, any other suitable clause, or any combination of suitable clauses, wherein the therapeutic target is NLRX1.
444. The method of clause 415, any other suitable clause, or any combination of suitable clauses, wherein the therapeutic target is PDE4.
445. The method of clause 415, any other suitable clause, or any combination of suitable clauses, wherein the therapeutic target is PSGL-1.
446. The method of clause 415, any other suitable clause, or any combination of suitable clauses, wherein the therapeutic target is RIPK1.
447. The method of clause 415, any other suitable clause, or any combination of suitable clauses, wherein the therapeutic target is S1P1.
448. The method of clause 415, any other suitable clause, or any combination of suitable clauses, wherein the therapeutic target is TLIA.
449. The method of clause 415, any other suitable clause, or any combination of suitable clauses, wherein the therapeutic target is TLR9.
450. The method of clause 415, any other suitable clause, or any combination of suitable clauses, wherein the therapeutic target is TNFa.
451. The method of clause 415, any other suitable clause, or any combination of suitable clauses, wherein the therapeutic target is TNFSF15.
452 The method of clause 415, any other suitable clause, or any combination of suitable clauses, wherein the therapeutic target is TPL2.
453. The method of clause 415, any other suitable clause, or any combination of suitable clauses, wherein the therapeutic target is TREM1.
454. The method of clause 415, any other suitable clause, or any combination of suitable clauses, wherein the therapeutic target is TYK2.
455. The method of clause 383, any other suitable clause, or any combination of suitable clauses, wherein the assessment comprises monitoring of mucosal lesions in the subject.
456. The method of clause 383, any other suitable clause, or any combination of suitable clauses, wherein the assessment comprises evaluation of inflammation in the subject.
457. The method of clause 456, any other suitable clause, or any combination of suitable clauses, wherein the evaluation of inflammation comprises analysis of quantity of inflammation.
458. The method of clause 456, any other suitable clause, or any combination of suitable clauses, wherein the evaluation of inflammation comprises analysis of an increase or a decrease in inflammation.
459. The method of clause 456, any other suitable clause, or any combination of suitable clauses, wherein the evaluation of inflammation comprises analysis of change in inflammation.
460. The method of clause 383, any other suitable clause, or any combination of suitable clauses, wherein the assessment comprises evaluation of a cell type in the subject. 461. The method of clause 460, any other suitable clause, or any combination of suitable clauses, wherein the evaluation comprises analysis of quantity of the cell type.
462. The method of clause 460, any other suitable clause, or any combination of suitable clauses, wherein the evaluation comprises analysis of an increase or a decrease of the cell type.
463. The method of clause 460, any other suitable clause, or any combination of suitable clauses, wherein the evaluation comprises analysis of a change in relative proportion of the cell type.
464. The method of clause 460, any other suitable clause, or any combination of suitable clauses, wherein the cell type is an inflammatory cell.
465. The method of clause 460, any other suitable clause, or any combination of suitable clauses, wherein the cell type is an immunogenic cell.
466. The method of clause 460, any other suitable clause, or any combination of suitable clauses, wherein the cell type is an enterocyte cell.
467. The method of clause 460, any other suitable clause, or any combination of suitable clauses, wherein the cell type is an enterocyte-related cell.
468. The method of clause 460, any other suitable clause, or any combination of suitable clauses, wherein the cell type is a lymphocyte cell.
469. The method of clause 460, any other suitable clause, or any combination of suitable clauses, wherein the cell type is a lymphocyte-related cell.
470. The method of clause 460, any other suitable clause, or any combination of suitable clauses, wherein the cell type is a T cell.
471. The method of clause 460, any other suitable clause, or any combination of suitable clauses, wherein the cell type is an NK cell.
472. The method of clause 460, any other suitable clause, or any combination of suitable clauses, wherein the cell type is a B cell.
473. The method of clause 460, any other suitable clause, or any combination of suitable clauses, wherein the cell type is a macrophage.
474. The method of clause 460, any other suitable clause, or any combination of suitable clauses, wherein the cell type is a neutrophil.
475. The method of clause 460, any other suitable clause, or any combination of suitable clauses, wherein the cell type is an endothelial cell.
476. The method of clause 460, any other suitable clause, or any combination of suitable clauses, wherein the cell type is a fibroblast.
477. The method of clause 383, any other suitable clause, or any combination of suitable clauses, wherein the assessment is a prognosis of therapeutic response to a medical intervention in the subject.
478. The method of clause 477, any other suitable clause, or any combination of suitable clauses, wherein the medical intervention is a drug therapy.
479. The method of clause 478, any other suitable clause, or any combination of suitable clauses, wherein the drug therapy is an IBD drug therapy.
480. The method of clause 478, any other suitable clause, or any combination of suitable clauses, wherein the drug therapy is a UC drug therapy.
481. The method of clause 478, any other suitable clause, or any combination of suitable clauses, wherein the drug therapy is a CD drug therapy.
482. The method of clause 478, any other suitable clause, or any combination of suitable clauses, wherein the drug therapy comprises an anti-inflammatory drug.
483. The method of clause 478, any other suitable clause, or any combination of suitable clauses, wherein the drug therapy comprises an immune system suppressing drug.
484. The method of clause 478, any other suitable clause, or any combination of suitable clauses, wherein the drug therapy comprises a biologic.
485. The method of clause 478, any other suitable clause, or any combination of suitable clauses, wherein the drug therapy comprises an antibiotic.
486. The method of clause 477, any other suitable clause, or any combination of suitable clauses, wherein the medical intervention is a colonoscopy.
487. The method of clause 383, any other suitable clause, or any combination of suitable clauses, wherein the assessment is a determination of therapeutic effectiveness of a medical intervention in the subject.
488. The method of clause 487, any other suitable clause, or any combination of suitable clauses, wherein the medical intervention is a drug therapy.
489. The method of clause 488, any other suitable clause, or any combination of suitable clauses, wherein the drug therapy is an IBD drug therapy.
490. The method of clause 488, any other suitable clause, or any combination of suitable clauses, wherein the drug therapy is a UC drug therapy.
491. The method of clause 488, any other suitable clause, or any combination of suitable clauses, wherein the drug therapy is a CD drug therapy.
492. The method of clause 488, any other suitable clause, or any combination of suitable clauses, wherein the drug therapy comprises an anti-inflammatory drug.
493. The method of clause 488, any other suitable clause, or any combination of suitable clauses, wherein the drug therapy comprises an immune system suppressing drug.
494. The method of clause 488, any other suitable clause, or any combination of suitable clauses, wherein the drug therapy comprises a biologic.
495. The method of clause 488, any other suitable clause, or any combination of suitable clauses, wherein the drug therapy comprises an antibiotic.
496. The method of clause 487, any other suitable clause, or any combination of suitable clauses, wherein the medical intervention is a colonoscopy.
497. The method of clause 383, any other suitable clause, or any combination of suitable clauses, wherein measuring the level of expression of nucleic acid biomarkers comprises nucleic acid extraction from the eukaryotic cells.
498. The method of clause 383, any other suitable clause, or any combination of suitable clauses, wherein measuring the level of expression of nucleic acid biomarkers comprises DNA extraction from the eukaryotic cells.
499. The method of clause 383, any other suitable clause, or any combination of suitable clauses, wherein measuring the level of expression of nucleic acid biomarkers comprises RNA extraction from the eukaryotic cells.
500. The method of clause 383, any other suitable clause, or any combination of suitable clauses, wherein measuring the level of expression of nucleic acid biomarkers comprises next generation sequencing.
501. The method of clause 383, any other suitable clause, or any combination of suitable clauses, wherein the method further comprises c) diagnosing disease in the subject.
502. The method of clause 501, any other suitable clause, or any combination of suitable clauses, wherein the disease is inflammatory bowel disease (IBD).
503. The method of clause 501, any other suitable clause, or any combination of suitable clauses, wherein the disease is ulcerative colitis (UC).
504. The method of clause 501, any other suitable clause, or any combination of suitable clauses, wherein the disease is Crohn's disease (CD).
505. The method of clause 383, any other suitable clause, or any combination of suitable clauses, wherein the method further comprises c) treating disease in the subject.
506. The method of clause 505, any other suitable clause, or any combination of suitable clauses, wherein the disease is inflammatory bowel disease (IBD).
507. The method of clause 505, any other suitable clause, or any combination of suitable clauses, wherein the disease is ulcerative colitis (UC).
508. The method of clause 505, any other suitable clause, or any combination of suitable clauses, wherein the disease is Crohn's disease (CD).
509. The method of clause 505, any other suitable clause, or any combination of suitable clauses, wherein treating disease comprises administering an IBD drug therapy to the subject.
510. The method of clause 505, any other suitable clause, or any combination of suitable clauses, wherein treating disease comprises administering a UC drug therapy to the subject.
511. The method of clause 505, any other suitable clause, or any combination of suitable clauses, wherein treating disease comprises administering a CD drug therapy to the subject.
512. The method of clause 505, any other suitable clause, or any combination of suitable clauses, wherein treating disease comprises administering an anti-inflammatory drug to the subject.
513. The method of clause 505, any other suitable clause, or any combination of suitable clauses, wherein treating disease comprises administering an immune system suppressing drug to the subject.
514. The method of clause 505, any other suitable clause, or any combination of suitable clauses, wherein treating disease comprises administering a biologic to the subject.
515. The method of clause 505, any other suitable clause, or any combination of suitable clauses, wherein treating disease comprises administering an antibiotic to the subject.

### EXAMPLES

### Example 1

### Analysis of seRNA in Crohn's Disease Subjects

Stool samples were collected from 68 individuals who were observed at routine clinic visits at up to three (3) time points prior to and after initiation of an advanced therapy. Stool samples underwent RNA extraction and sequencing using a custom capture panel (n=1,507 transcripts). Stool-derived eukaryotic RNA (seRNA) signatures were compared to CD activity index (CDAI) scores and endoscopies, when available. Random forest models were built to assess the ability to classify disease severity when compared to CDAI scores. Further, seRNA signatures were also used to assess expression of the therapy target and cell type abundance at various time points.

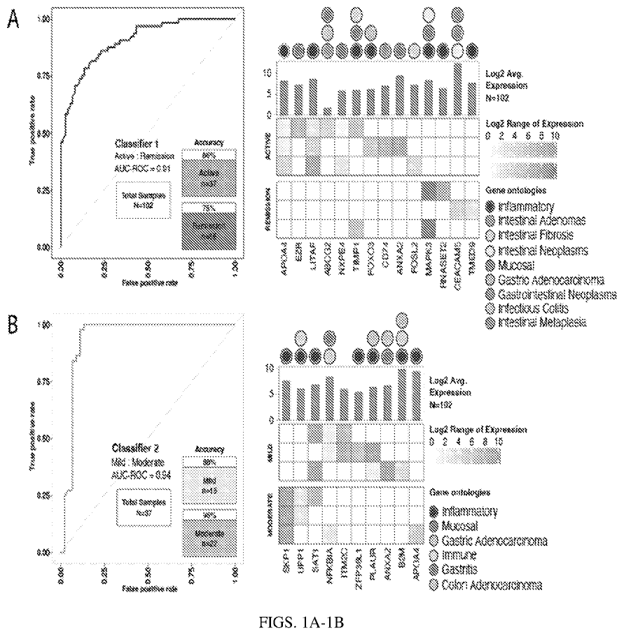

Across the 102 samples collected from 68 individuals, the random forest classifier successfully parsed individuals with active disease (n=37) relative to those in remission (n=65) with 81% accuracy. Machine learning models were developed using 5-fold internal cross validation. As shown in FIG. 1A, Classifier 1 predicted subjects with active disease (n=37) relative to subjects in remission (n=65) when compared to the Crohn's Disease Activity Index (CDAI). As shown in FIG. 1B, for subjects with active disease, Classifier 2 further parsed those with mild disease (n=16) relative to those with moderate disease (n=22). For each classifier, the top three biomarker signatures are shown. Each row in the heatmap indicates a composite biomarker which is composed of highlighted transcripts in that row. The shade of the highlight indicates transcript expression. Gene ontologies for each transcript are also provided.

A second classifier was employed on subjects with active (mild or moderate) disease (n=37). This classifier successfully parsed individuals with mild disease (n=15) from those with moderate disease (n=22) with 92% accuracy. For the 16 subjects with longitudinal data, seRNA signatures prior to treatment with either vedolizumab, ustekinumab, and infliximab, showed high expression of associated therapeutic targets (e.g., ITGA4/ITGB7 for vedolizumab, IL12A/IL12B/IL23A for ustekinumab, or TNF for infliximab) at TO for responders with a reduction in lymphocyte signatures during treatment.

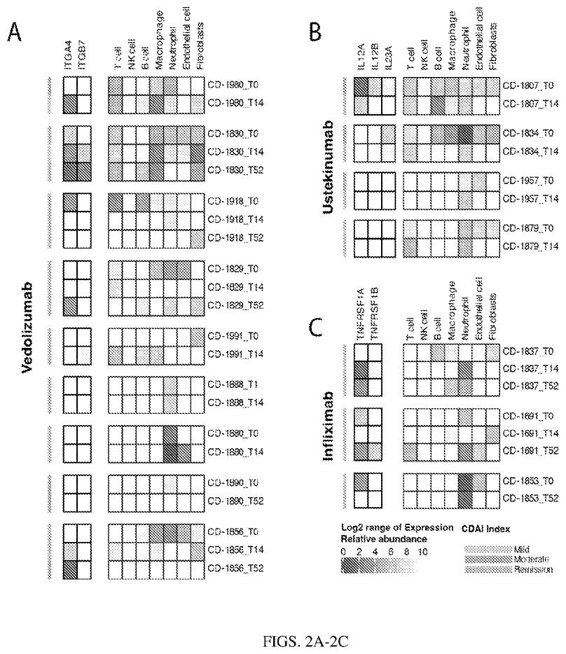

FIGS. 2A-2C display various subjects for treatment with vedolizumab, ustekinumab, and infliximab, respectively. For each subject, the first column of boxes indicates the expression of the transcript related to the therapy target, the pathway of the therapeutic target, or the receptor associated with the therapy. The second column of boxes provides expression of various lymphocytes or stromal cells (i.e., cell type deconvolution). For each subject, the number of rows corresponds to the number of longitudinal samples that were assessed, ordered such that the first box is the earliest time point and subsequent boxes are future time points chronologically. Target expression and cell type deconvolution are provided at multiple time points to show expression change during the course treatment. The darker shade of a particular box represents increased normalized expression.

### Example 2

### Alternate Analysis of seRNA in Crohn's Disease Subjects

Stool samples can be collected from Crohn's Disease subjects being evaluated for therapeutic treatment. For instance, subjects can be randomized into a drug treatment group and a placebo (control) group. Subjects can be evaluated for efficacy, safety, and/or tolerability of the drug treatment in comparison to the placebo according to the instant example.

Patients can be evaluated for clinical response, endoscopic response, clinical outcome, and/or histologic assessments. During the study, stool samples can be collected from subjects prior to, during, and/or following completion of the drug or placebo treatment.

The stool samples can undergo seRNA extraction and sequencing to assess transcriptomic changes associated with drug treatment and therapeutic response. For instance, stool samples collected from subjects can be subjected to total RNA extraction and/or next-generation sequencing based on previously provided methods. The sequencing data can be compared to one or more of clinical response, endoscopic response, clinical outcome, and/or histologic assessments

### Example 3

### Analysis of seRNA in Ulcerative Colitis Subjects

Stool samples were obtained from 15 subjects with active ulcerative colitis (UC) and 15 healthy volunteers. Diagnosis of UC was confirmed by a recent endoscopy or based on a patient having no history of disease. Stool samples were collected and frozen with or without stabilization buffer prior to analysis. Samples where thawed and underwent parallel assessment of both proteins and nucleic acids. Protein assessment was performed via an ELISA assay to quantify human calprotectin in the sample. Each stool sample underwent two replicates of ELISA calprotectin quantification. Nucleic acid assessment was performed via eukaryotic RNA extraction, RNA quality control assessment, library preparation, hybridization capture, and whole transcriptome sequencing.

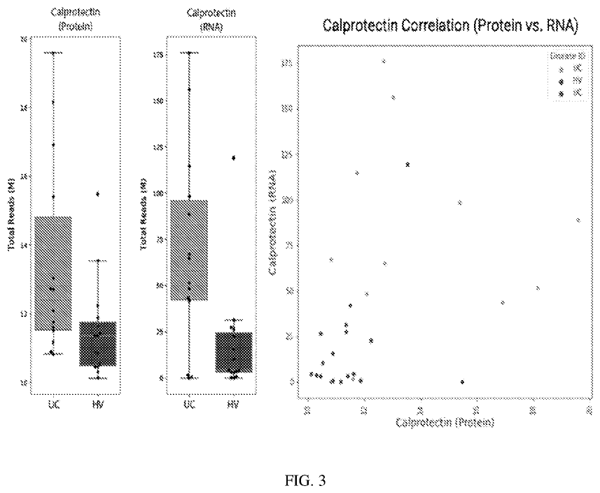

Log2 of the average concentration of the ELISA calprotectin assay was compared to total S100A8 concentration or S100A9 concentration (transcripts of calprotectin subunits) normalized to total read counts. FIG. 3 shows protein-based calprotectin measurements (ELISA assay) compared to RNA-based calprotectin measurements (whole transcriptome sequencing). As shown in FIG. 3, a significant increase was observed in both the protein- and RNA-based calprotectin measurements when comparing individuals with Ulcerative Colitis (UC) relative to healthy volunteers. There was a correlation between protein- and RNA-based calprotectin measurements. Sensitivity for RNA-based measurements was correlated with ability to measure housekeeping transcript concentrations and total readcounts. For example, UC subjects with low read counts for GAPDH (red dots on scatterplot) had lower normalized RNA-based calprotectin measurements.

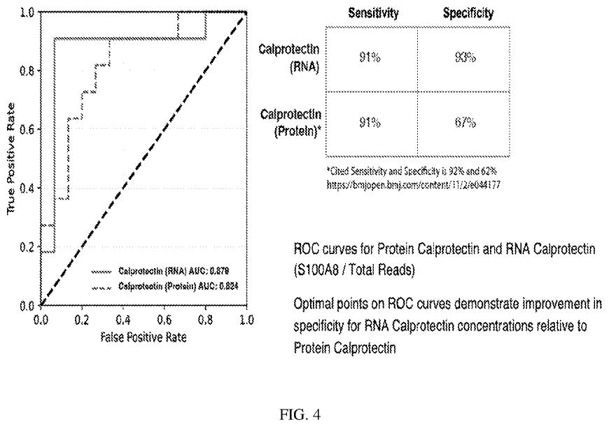

FIG. 4 shows a plot of the receiver operator characteristic (ROC) in which the protein-based method demonstrated a reduced area under the curve (AUC) relative to the RNA-based method (0.824 versus 0.879). When evaluating the optimal point on the ROC AUC for both the protein-based and RNA-based calprotectin measurements, the RNA-based measurements demonstrated a higher level of specificity.

### Example 4

### RNA and FIT Analysis of Patients with IBD

Stool samples were collected and fecal immunochemical testing was performed on 12 individuals with inflammatory bowel disease prior to undergoing a standard-of-care colonoscopy. Stool samples were shipped in the mail at ambient temperature for up to 96 hours. Stool samples underwent RNA extraction, quality assessment, and digital droplet PCR. Eight (8) RNA transcripts were queried to assess the presence of colorectal cancer and advanced adenomas. These transcripts, These transcripts, demographic information, and FIT results were assessed by an algorithm to determine a positive or negative result. Of the 12 subjects, 3 had other precancerous lesions on colonoscopy, 7 had hyperplastic polyps on colonoscopy, and 2 had no findings on colonoscopy. For the 3 subjects with adenomas, 1 was detected as positive with the RNA-FIT result. For the 7 subjects with hyperplastic polyps, 4 were detected as positive with the RNA-FIT result. For the two patients with no findings on colonoscopy, both were detected as negative with the RNA-FIT result.

1. A method of assessing disease in a subject, the method comprising:
measuring the level of expression of one or more stool-derived eukaryotic nucleic acid biomarkers selected from the biomarkers described herein in eukaryotic nucleic acid extracted from a stool sample from the subject,
wherein the measured expression level of the one or more stool-derived eukaryotic nucleic acid biomarkers in the stool sample indicates the assessment of disease in the subject.

2. The method of claim 1, wherein the method comprises comparing the measured expression level of the one or more stool-derived eukaryotic nucleic acid biomarkers in the stool sample with the measured expression level of the one or more stool-derived eukaryotic nucleic acid biomarkers in a control, wherein a difference in the measured expression level of the one or more stool-derived eukaryotic nucleic acid biomarkers in the stool sample relative to the measured expression level of the one or more stool-derived eukaryotic nucleic acid biomarkers in the control indicates the assessment of disease in the subject.

3. The method of claim 1, wherein the nucleic acid is DNA.

4. The method of claim 1, wherein the nucleic acid is RNA.

5. The method of claim 1, wherein the disease is inflammatory bowel disease (IBD).

6. The method of claim 1, wherein the disease is ulcerative colitis (UC).

7. The method of claim 1, wherein the disease is Crohn's disease (CD).

8. The method of claim 1, wherein the assessment of disease comprises an assessment of disease activity in the subject.

9. The method of claim 1, wherein the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 1.

10. The method of claim 1, wherein the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 2.

11. The method of claim 1, wherein the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 3.

12. The method of claim 1, wherein the stool-derived eukaryotic nucleic acid biomarkers comprise 2, 3, 4, 5, 6, 7, 8, 9, 10, 11, 12, 13, 14, 15, 16, 17, 18, 19, 20, 21, 22, 23, 24, 25, 26, 27, 28 or 29 biomarkers selected from the biomarkers listed in Table 4.

13. The method of claim 1, wherein the stool-derived eukaryotic nucleic acid biomarkers comprise a combination of biomarkers listed in Table 5.

14. The method of claim 1, wherein the one or more stool-derived eukaryotic nucleic acid biomarkers comprise stool-derived eukaryotic RNA biomarkers, wherein the stool-derived eukaryotic RNA biomarkers comprise one or more therapeutic targets.

15. The method of claim 1, wherein the assessment comprises evaluation of inflammation in the subject.

16. The method of claim 1, wherein the assessment comprises evaluation of a cell type in the subject.

17. The method of claim 1, wherein the method further comprises diagnosing disease in the subject, wherein the disease is inflammatory bowel disease (IBD).

18. The method of claim 1, wherein the method further comprises diagnosing disease in the subject, wherein the disease is ulcerative colitis (UC).

19. The method of claim 1, wherein the method further comprises diagnosing disease in the subject, wherein the disease is Crohn's disease (CD).

20. A method of predicting therapeutic response to a disease in a subject, the method comprising:
measuring the level of expression of one or more stool-derived eukaryotic nucleic acid biomarkers selected from the biomarkers described herein in eukaryotic nucleic acid extracted from a stool sample from the subject,
wherein the measured expression level of the one or more stool-derived eukaryotic nucleic acid biomarkers in the stool sample indicates therapeutic response to the disease in the subject.

21.-79. (canceled)
